SEQUENCE LISTING (2 sequences)

SEQ ID NO: 1 - DNA, 3700 bp, Homo sapiens
ctgtttcttggtttgtgtctctgctgcaagtccaaggagctggggcaataccttgagtctgggttcttcgtccccagggacctgggggagccccagcaatgctcagggaaaggggagagcaaagtgtggggttggttctctctagtggtcagtgttggaactgcatccagctgactcaggctgacccaggagtcctcagcagaagtggaattcaggactgaatcgtgctcacaacccccacaatctattggctgtgcttggccccttttcccaacacacacattctgtctggtgggtggaggttaaacatgcggggaggaggaaagggataggatagagaatgggatgtggtcggtagggggtctcaaggactggctatcctgacatccttctccgcgttcaggttggccaccatggcctgcggccagagggcacccacgtgacccttaaagagaggacaagttgggtggtatctctggctgacactctgtgcacaaccctcacaacactggtgacggtgggaagggaaagatgacaagccagggggcatgatcccagcatgtgtgggaggagcttctaaattatccattagcacaagcccgtcagtggccccatgcataaatgtacacagaaacaggtgggggcaacagtgggagagaaggggccagggtataaaaagggcccacaagagaccagctcaaggatcccaaggcccaactccccgaaccactcagggtcctgtggacagctcacctagcggcaatggctacaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgtaagctcttggggaatgggtgcgcatcaggggtggcaggaaggggtgactttcccccgctgggaaataagaggaggagactaaggagctcagggtttttcccgaagcgaaaatgcaggcagatgagcacacgctgagtgaggttcccagaaaagtaacaatgggagctggtctccagcgtagaccttggtgggcggtccttctcctaggaagaagcctatatcccaaaggaacagaagtattcattcctgcagaacccccagacctccctctgtttctcagagtctattccgacaccctccaacagggaggaaacacaacagaaatccgtgagtggatgccttctccccaggcggggatgggggagacctgtagtcagagcccccgggcagcacagccaatgcccgtccttcccctgcagaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatgggggtgagggtggcgccaggggtccccaatcctggagccccactgactttgagagctgtgttagagaaacactgctgccctctttttagcagtcaggccctgacccaagagaactcaccttattcttcatttcccctcgtgaatcctccaggcctttctctacaccctgaaggggagggaggaaaatgaatgaatgagaaagggagggaacagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattcgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctgccatgcccaaccggtgggacatttgagttgcttgcttggcactgtcctctcatgcgttgggtccactcagtagatgcctgttgaattcctgggcctagggctgtgccagctgcctcgtcccgtcaccttctggcttcttctctccctccatatcttagctgttttcctcatgagaatgttccaaattcgaaatttctatttaaccattatatatttacttgtttgctattatctctgcccccagtagattgttagctccagaagagaaaggatcatgtcttttgcttatctagatatgcccatctgcctggtacaatctctggcacatgttacaggcaacaactacttgtggaattggtgaatgcatgaatagaagaatgagtgaatgaatgaatagacaaaaggcagaaatccagcctcaaagaacttacagtctggtaagaggaataaaatgtctgcaaatagccacaggacaggtcaaaggaaggaggggctatttccagctgagggcaccccatcaggaaagcaccccagacttcctacaactactagacacatctcgatgcttttcacttctctatcaatggatcgtctccctggagaataatccccaaagtgaaattacttagcacgtccagttaggtagatccttgtgtacttcttggttgttcagagatcatcaaccagtgcaaacaatccccccatcaatacacagcagtgcctgcccctctccccccgaggtcttccgaggcccttcctccgtgcctgaaccccctggacatatcatatggcaaactgaagtgtccaacgagatataggaagtgaaacacgatgtacactgaaacgtgcaatacaaatatgcagcatgaagtgcctcggttcactaacccgagctacgctgggtgcttcttttctaccactttcctta

SEQ ID NO: 2 - protein, 191 aa, Homo sapiens [sequence omitted]

(19) United States
(12) Patent Application Publication
(10) Pub. No.: US 2005/0233417 A1
(43) Pub. Date: Oct. 20, 2005

(54) Growth hormone variations in humans and their uses

(76) Inventors: David Neil Cooper, Cardiff (GB); Ann Marie Procter, Cardiff (GB); John Gregory, Cardiff (GB); David Stuart Millar, Cardiff (GB); Mark Lewis, Cardiff (GB); Angeles Ulied, Barcelona (ES)

Correspondence Address: QUARLES & BRADY LLP, 411 E. WISCONSIN AVENUE, SUITE 2040, MILWAUKEE, WI, 53202-4497, US

(21) Appl. No.: 10/495,235

(22) PCT Filed: Nov. 12, 2002
(86) PCT No.: PCT/GB02/05112

§ 371 Date: Mar. 21, 2005

(30) Foreign Application Priority Data
Nov. 12, 2001 (GB) 0127214.5
Nov. 14, 2001 (GB) 0127328.3

(51) Int. Cl. C07K 14/61; C07H 21/04; C12N 15/09

(52) U.S. Cl. 435/69.4; 435/320.1; 435/325; 530/399; 536/23.5; 514/12

(57) ABSTRACT

The present invention relates to naturally-occurring growth hormone mutations; to a method for detecting them and their use in screening patients for growth hormone irregularities or for producing variant proteins suitable for treating such irregularities. In one aspect there is disclosed variants of GH1, selected from the group consisting of: (a) (i) +480 C→T; (ii) +446 C→T; (iii) +1491 C→G; (iv) −60 G→A; (v) −40 to −39 GG→CT; (vi) −360 A→G; and (vii) +748 A→G (where figures relate to GH1 nucleotide position number, counting from TSS); (b) a sequence substantially homologous to or that hybridises to sequence (a) under stringent conditions; (c) a sequence substantially homologous to or that hybridises to the sequences (a) or (b) but for degeneracy of the genetic code; and (d) an oligonucleotide specific for any of the sequences (a), (b) or (c) above.

The present invention relates to naturally-occurring growth hormone mutations; to a method for detecting them and their use in screening patients for growth hormone irregularities or for producing variant proteins suitable for treating such irregularities.

That human stature was influenced by inherited factors was understood more than a century ago. Although familial short stature, with its normally recessive mode of inheritance, was recognised as early as 1912, it was a further quarter century before such families came to be properly documented in the scientific literature. The recognition that recessively inherited short stature was commonly associated with isolated growth hormone (GH) deficiency only came in 1966.

Short stature associated with GH deficiency has been estimated to occur with an incidence of between 1/4000 and 1/10000 live births. Most of these cases are both sporadic and idiopathic, but between 5 and 30% have an affected first-degree relative consistent with a genetic aetiology for the condition. Confirmation of the genetic aetiology of GH deficiency came from the molecular genetic analysis of familial short stature and the early demonstration of mutational lesions in the pituitary-expressed growth hormone (GH1) genes of affected individuals. Familial short stature may also be caused by mutation in a number of other genes (eg POU1F1, PROP1 and GHRHR) and it is important to distinguish these different forms of the condition.

Growth hormone (GH) is a multifunctional hormone that promotes post-natal growth of skeletal and soft tissues through a variety of effects. Controversy remains as to the relative contribution of direct and indirect actions of GH. On one hand, the direct effects of GH have been demonstrated in a variety of tissues and organs, and GH receptors have been documented in a number of cell types. On the other hand, a substantial amount of data indicates that a major portion of the effects of GH are mediated through the actions of GH-dependent insulin-like growth factor I (IGF-I). IGF-1 is produced in many tissues, primarily the liver, and acts through its own receptor to enhance the proliferation and maturation of many tissues, including bone, cartilage, and skeletal muscle. In addition to promoting growth of tissues, GH has also been shown to exert a variety of other biological effects, including lactogenic, diabetogenic, lipolytic and protein anabolic effects, as well as sodium and water retention.

Adequate amounts of GH are needed throughout childhood to maintain normal growth. Newborns with GH deficiency are usually of normal length and weight. Some may have a micropenis or fasting hypoglycemia in conjunction with low linear postnatal growth, which becomes progressively retarded with age. In those with isolated growth hormone deficiency (IGHD), skeletal maturation is usually delayed in association with their height retardation. Truncal obesity, facial appearance younger than expected for their chronological age and delayed secondary dentition are often present. Skin changes similar to those seen in premature ageing may be seen in affected adults.

Familial IGHD comprises several different disorders with characteristic modes of inheritance. Those forms of IGHD known to be associated with defects at the GH1 gene locus are shown in Table 1 together with the different types of underlying lesion so far detected.

TABLE 1
Classification of inherited disorders involving the GH1 gene

| | | Types of gene | | |
|---|---|---|---|---|
| | Mode of | lesion | GH | |
| Disorder | inheritance | responsible | protein | Deficiency state |
| IGHD IA | Autosomal | Gross deletions, | Absent | Severe short stature. |
| | recessive | micro-deletions, | | Anti-GH antibodies often |
| | | nonsense mutations | | produced upon GH |
| | | | | treatment, resulting in |
| | | | | poor response thereto. |
| IGHD IB | Autosomal | Splice site | Deficient | Short stature. Patients |
| | recessive | mutations | | usually respond well to |
| | | | | exogenous GH. |
| IGHD II | Autosomal | Splice site and | Deficient | Short stature. Patients |
| | dominant | intronic mutations, | | usually respond well to |
| | | missense mutations | | exogenous GH. |

The characterisation of these lesions has helped to provide explanations for the differences in clinical severity, mode of inheritance and propensity to antibody formation in response to exogenously administered GH, between these forms of IGHD. Most cases are sporadic and are assumed to arise from cerebral insults or defects that include cerebral oedema, chromosomal anomalies, histiocytosis, infections, radiation, septo-optic dysplasia, trauma, or tumours affecting the hypothalamus or pituitary. Magnetic resonance imaging examinations detect hypothalamic or pituitary anomalies in about 12% of patients who have IGHD.

Although short stature, delayed ‘height velocity’ or growth velocity, and delayed skeletal maturation are all seen with GH deficiency, none of these is specific for this disorder; other systemic diseases may result in such symptoms. Throughout this specification, ‘height velocity’ and growth velocity are both to be construed as meaning the rate of change of the subject's or patient's height, such as is measured in centimetres per year.

Stimulation tests to demonstrate GH deficiency use L-Dopa, insulin-induced hypoglycaemia, arginine, insulin-arginine, clonidine, glucagon or propranolol. Inadequate GH peak responses (usually <7-10 ng/mL) differ from test to test. Testing for concomitant deficiencies of LH, FSH, TSH and ACTH should be performed to determine the extent of pituitary dysfunction and to plan optimal treatment.

Recombinant-derived GH is available worldwide and is administered by subcutaneous injection. To obtain an optimal outcome, children with IGHD are usually started on replacement therapy as soon as their diagnosis is established. The initial dosage of recombinant GH is based on body weight or surface area, but the exact amount used and the frequency of administration may vary between different protocols. The dosage increases with increasing body weight to a maximum during puberty. Thereafter, GH treatment should be temporarily discontinued while the individual's GH secretory capacity is re-evaluated. Those with confirmed GH deficiency receive a lower dose of exogenous GH during adult life.

Conditions that are treated with GH include (i) those in which it has proven efficacy and (ii) a variety of others in which its use has been reported but not accepted as standard practice. Disorders in which GH treatment has proven efficacy include GH deficiency, either isolated or in association with combined pituitary hormone deficiency (CPHD) and Turner syndrome. The clinical responses of individuals with the first two disorders to GH replacement therapy varies depending on: (i) the severity of the GH deficiency and its adverse effects on growth, the age at which treatment is begun, weight at birth, current weight and dose of GH; and (ii) recognition and response to treatment of associated deficiencies such as thyroid hormone deficiency; and (iii) whether treatment is complicated by the development of anti-GH antibodies. The outcome of treatment for individuals with Turner syndrome varies with the severity of their short stature, their chromosomal complement, and the age at which treatment was begun.

Additional disorders in which the use of GH has been reported include treatment of certain skeletal dysplasias such as achondroplasia, Prader-Willi syndrome, growth suppression secondary to exogenous steroids or in association with chronic inflammatory diseases such as rheumatoid arthritis, in chronic renal failure, extreme idiopathic short stature, Russell-Silver syndrome, and intrauterine growth retardation.

The characterisation of familial IGHD at the molecular genetic level is important for several reasons. The identity of the locus involved will indicate not only the likely severity of growth retardation but, more importantly, the appropriateness or otherwise of the various therapeutic regimens now available. Further, detection of the underlying gene lesions serves to confirm the genetic aetiology of the condition. It may also have prognostic value in predicting (i) the severity of growth retardation and (ii) the likelihood of anti-GH antibody formation subsequent to GH treatment. In some instances, knowledge of the pathological lesion(s) can also help to explain an unusual mode of inheritance of the disorder and is therefore essential for the counselling of affected families. Finally, the characterisation of the mutational lesions responsible for cases of IGHD manifesting a dysfunctional (as opposed to a non-functional) GH molecule could yield new insights into GH structure and function.

At the cellular level, a single GH molecule binds two GH receptor molecules (GHR) causing them to dimerise. Dimerisation of the two GH-bound GHR molecules is believed to be necessary for signal transduction, which is associated with the tyrosine kinase JAK2. It has been suggested that the diverse effects of GH may be mediated by a single type of GHR molecule that can possess different cytoplasmic domains or phosphorylation sites in different tissues. When activated by JAK2, these differing cytoplasmic domains can lead to distinct phosphorylation pathways, one for growth effects and others for various metabolic effects.

GH is a 22 kDa protein secreted by the somatotroph cells of the anterior pituitary. X-ray crystallographic studies have shown GH to comprise a core of two pairs of parallel alpha helices arranged in an up-up-down-down fashion. This structure is stabilised by two intra-molecular disulphide linkages (Cys53-Cys165 and Cys182-Cys 189). Two growth hormone receptor (GHR) molecules bind to two structurally distinct sites on the GH molecule, a process which proceeds sequentially by GHR binding first at site 1 and then at site 2. The binding of GHR to GH potentiates dimerisation of the GHR molecules.

Scanning mutagenesis studies of the GH molecule have yielded a picture of the binding interactions between GH and its receptor whilst site-directed mutagenesis has been used to probe the function of specific residues. Thus, substitution of Gly120 (in the third alpha helix of human GH) by Arg results in the loss of GHR binding to site 2 thereby blocking GHR dimerisation. Similarly, residue Phe44 of the human GH protein is important for binding the prolactin receptor. Finally, residues Asp115, Gly119, Ala122 and Leu123 have been shown to be critical for the growth enhancing potential of the murine GH molecule.

Interaction of the dimerised GHR with the intracellular tyrosine protein kinase JAK2 leads to tyrosine phosphorylation of downstream signal transduction molecules, stimulation of mitogen-activated protein (MAP) kinases and induction of signal transducers and activators of transcription (STAT proteins). In this way, GH is able to influence the expression of multiple genes through a number of different signalling pathways.

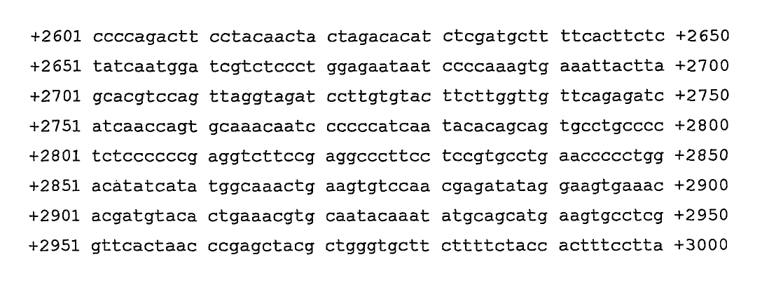

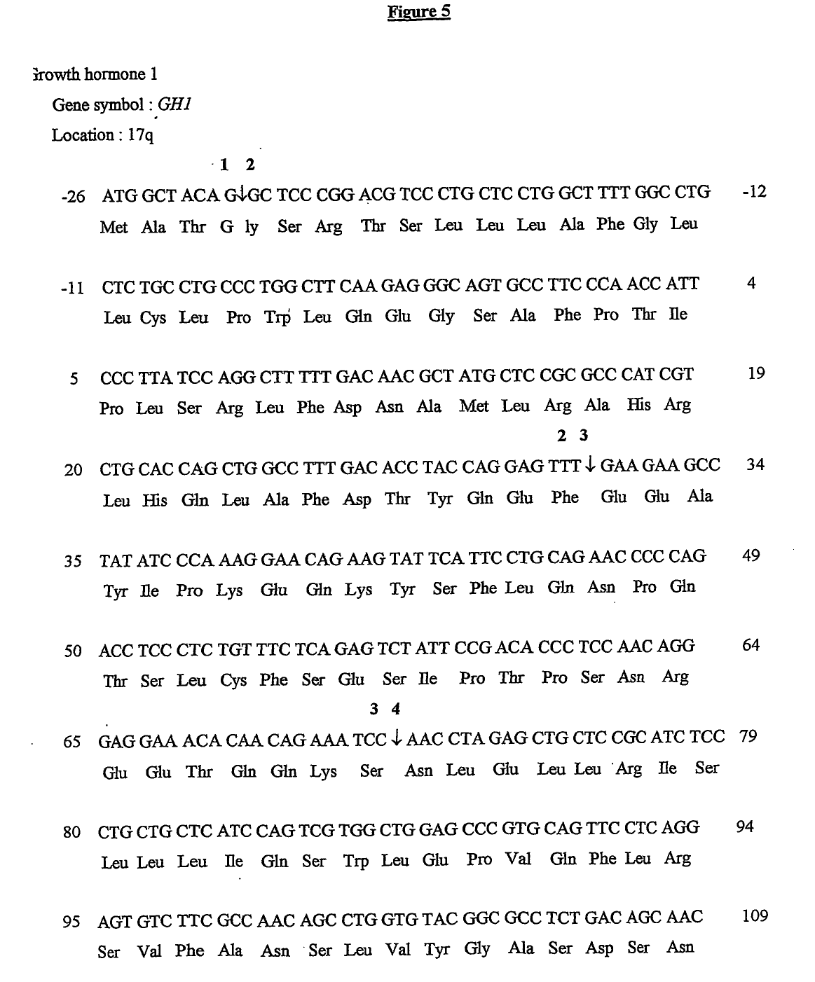

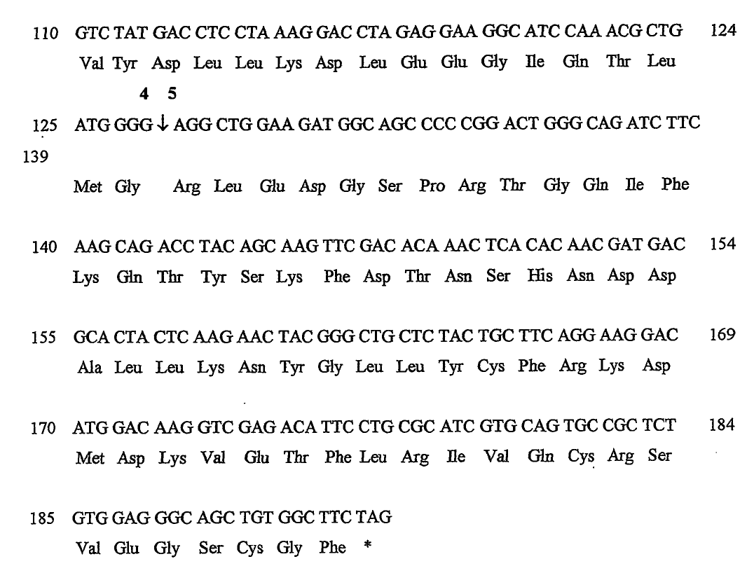

Several different GH isoforms are generated from expression of the GH1 gene (GH1 reference sequence is shown in FIG. 4). In 9% of GH1 transcripts, exon 2 is spliced to an alternative acceptor splice site 45 bp into exon 3, thereby deleting amino acid residues 32 to 46 and generating a 20 kDa isoform instead of the normal 22 kDa protein. This 20 kDa isoform appears to be capable of stimulating growth and differentiation. The factors involved in determining alternative acceptor splice site selection are not yet characterised but are clearly of a complex nature. A 17.5 kDa isoform, resulting from the absence of codons 32 to 71 encoded by exon 3, has also been detected in trace amounts in pituitary tumour tissue. Splicing products lacking either exons 3 and 4 or exons 2, 3 and 4 have been reported in pituitary tissue but these appear to encode inactive protein products. A 24 kDa glycosylated variant of GH has also been described. The amino acid sequence of the major 22 kDa isoform is presented in FIG. 5, which shows the nucleotide sequence of the GH1 gene coding region and amino acid sequence of the protein including the 26 amino acid leader peptide. Lateral numbers refer to amino acid residue numbering. Numbers in bold flanking vertical arrows specify the exon boundaries. The termination codon is marked with an asterisk.

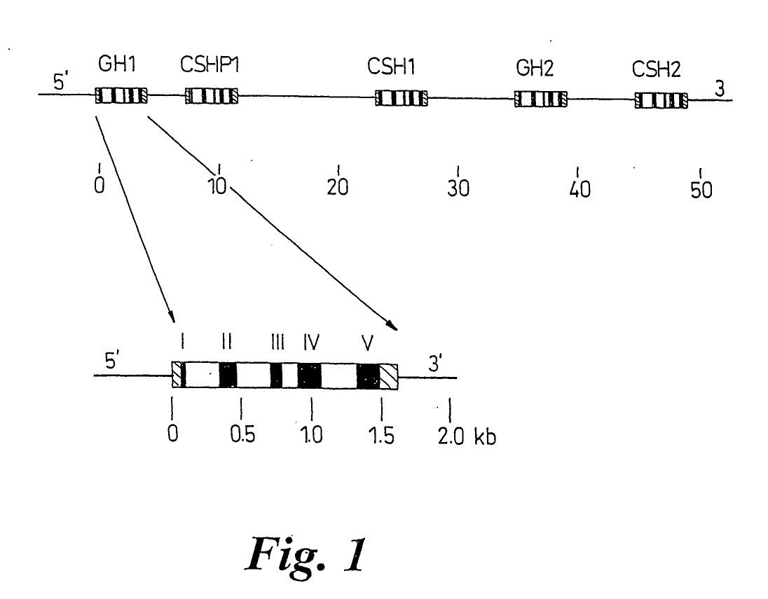

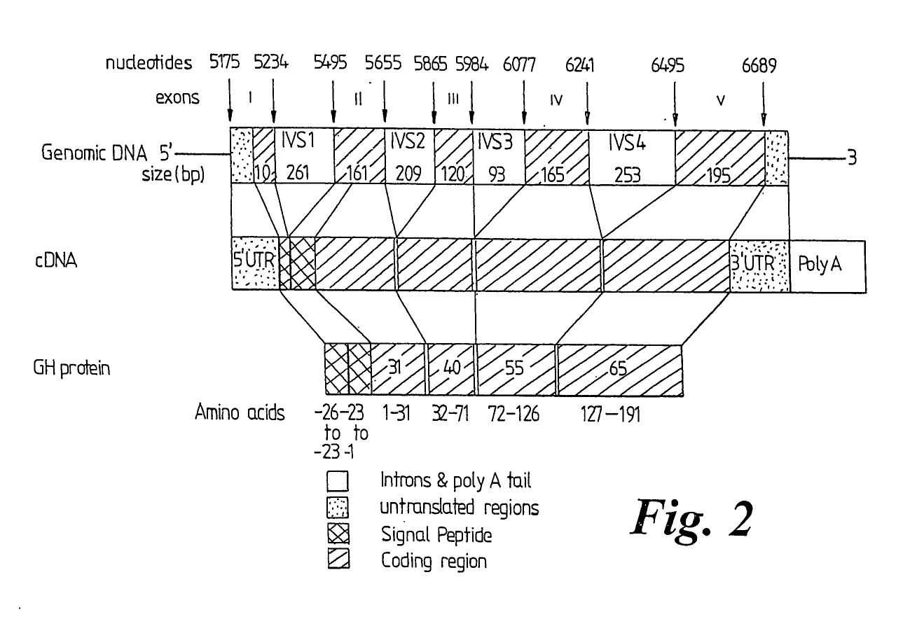

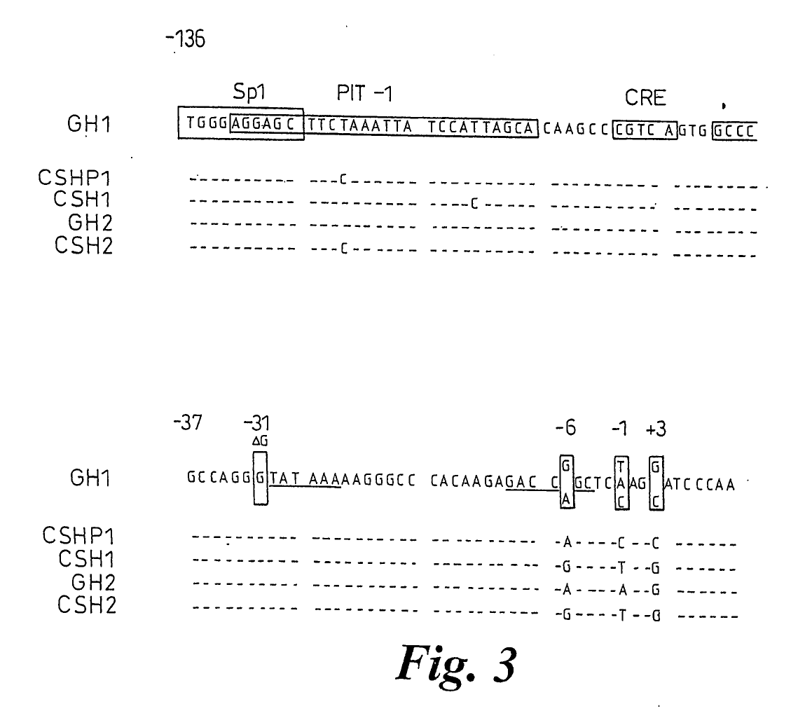

The gene encoding pituitary growth hormone (GH1) is located on chromosome 17q23 within a cluster of five related genes (FIG. 1). This 66.5 kb cluster has now been sequenced in its entirety [Chen et al. Genomics 4 479-497 (1989) and see FIG. 4]. The other loci present in the growth hormone gene cluster are two chorionic somatomammotropin genes (CSH1 and CSH2), a chorionic somatomammotropin pseudogene (CSHP1) and a growth hormone gene (GH2). These genes are separated by intergenic regions of 6 to 13 kb in length, lie in the same transcriptional orientation, are placentally expressed and are under the control of a downstream tissue-specific enhancer. The GH2 locus encodes a protein that differs from the GH1-derived growth hormone at 13 amino acid residues. All five genes share a very similar structure with five exons interrupted at identical positions by short introns, 260 bp, 209 bp, 92 bp and 253 bp in length in the case of GH1 (FIG. 2).

Exon 1 of the GH1 gene contains 60 bp of 5′ untranslated sequence (although an alternative transcriptional initiation site is present at −54), codons −26 to −24 and the first nucleotide of codon −23 corresponding to the start of the 26 amino acid leader sequence. Exon 2 encodes the rest of the leader peptide and the first 31 amino acids of mature GH. Exons 3-5 encode amino acids 32-71, 72-126 and 127-191, respectively. Exon 5 also encodes 112 bp 3′ untranslated sequence culminating in the polyadenylation site. An Alu repetitive sequence element is present 100 bp 3′ to the GH1 polyadenylation site. Although the five related genes are highly homologous throughout their 5′ flanking and coding regions, they diverge in their 3′ flanking regions.

The GH1 and GH2 genes differ with respect to their mRNA splicing patterns. As noted above, in 9% of GH1 transcripts, exon 2 is spliced to an alternative acceptor splice site 45 bp into exon 3 to generate a 20 kDa isoform instead of the normal 22 kDa. The GH2 gene is not alternatively spliced in this fashion. A third 17.5 kDa variant, which lacks the 40 amino acids encoded by exon 3 of GH1, has also been reported.

The CSH1 and CSH2 loci encode proteins of identical sequence and are 93% homologous to the GH1 sequence at the DNA level. By comparison with the CSH gene sequences, the CSHP1 pseudogene contains 25 nucleotide substitutions within its “exons” plus a G→A transition in the obligate +1 position of the donor splice site of intron 2 that partially inactivates its expression.

A number of biallelic restriction fragment length polymorphisms (RFLPs) have been reported within the GH gene region. Five of these (two BglII, two MspI, one HincI) occur in Caucasians and Blacks whereas a further BamHI polymorphism occurs predominantly in Blacks. Strong linkage disequilibrium has been observed between these polymorphisms consistent with the relatively recent evolutionary origin of the gene cluster. The HincII and BamHI polymorphisms occur immediately 5′ to the GH1 gene. An RsaI polymorphism occurs in the GH1 promoter region resulting from an A/G dimorphism at nucleotide −75 whilst a relatively frequent Sph1 polymorphism remains to be fully characterised. A highly informative (83% heterozygosity) variable number repeat polymorphism has been located some 19 kb 3′ to the GH1 gene; formatted for PCR, the 18 distinct alleles of this polymorphism can be distinguished by fragment size (201 to 253 bp).

Finally, the GH1 gene promoter/5′-untranslated region has been found to exhibit a very high level of sequence polymorphism with 17 variant nucleotides within a 570 bp stretch (Table 2A):

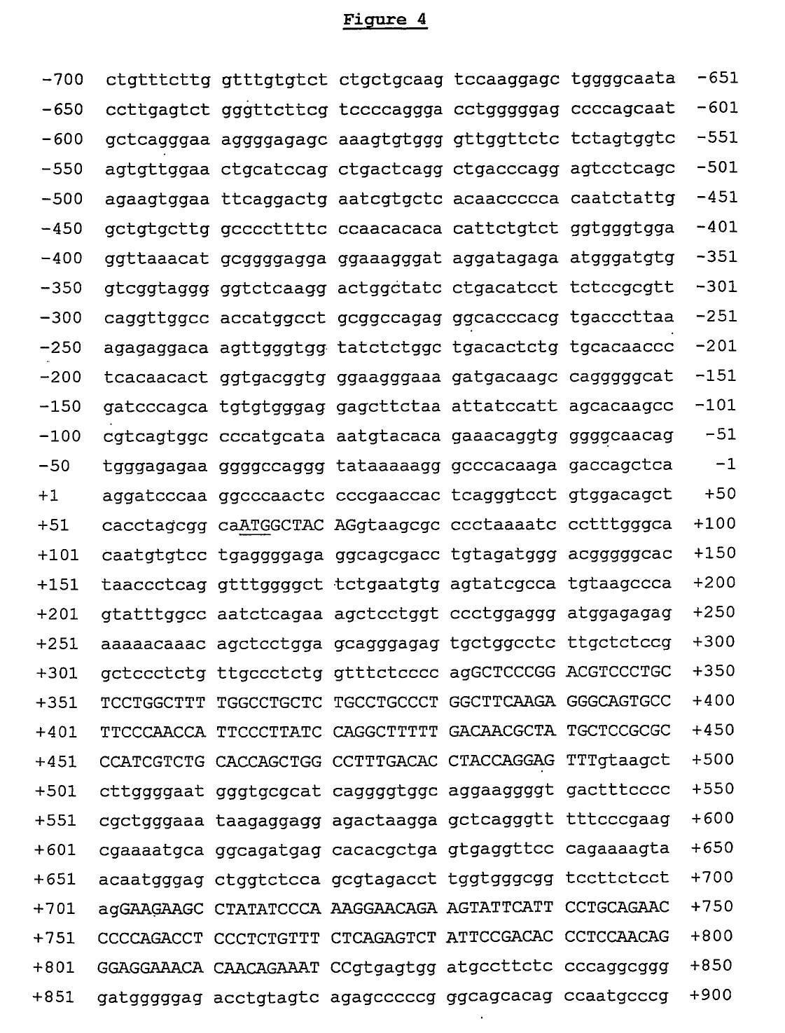

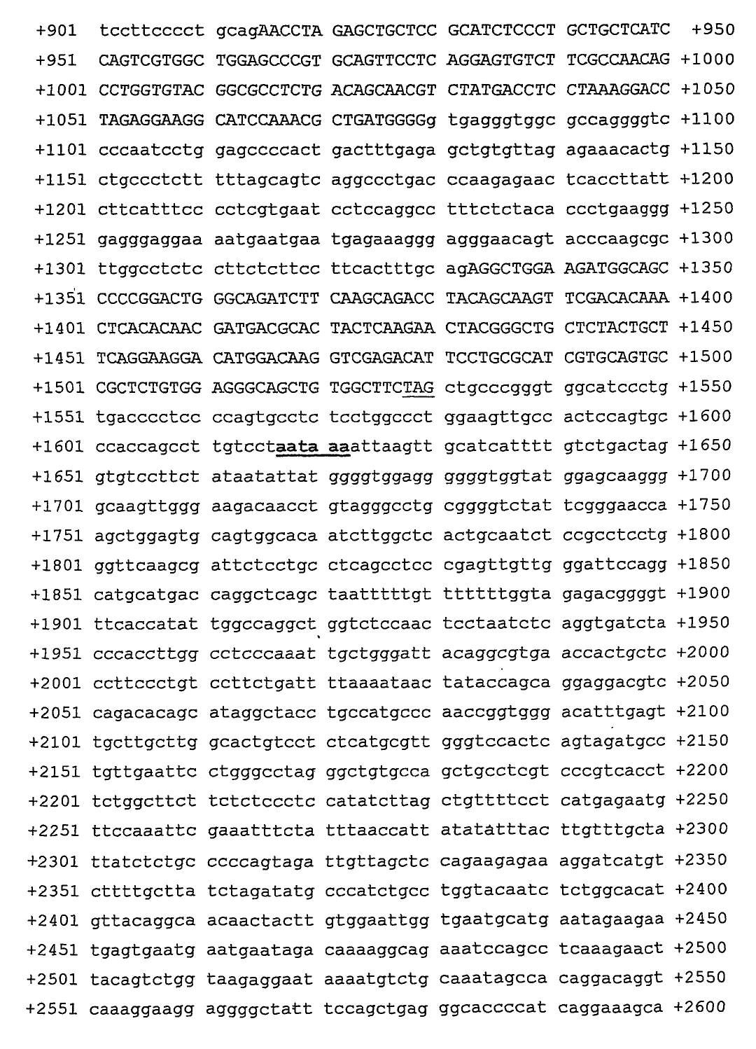

TABLE 2A
Known polymorphisms in the human GH1 gene promoter/5′ untranslated
region [after Giordano et al Human Genetics 100 249-255
(1997) and Wagner et al Eur. J. Endocrinol. 137 474-481].
(FIG. 3).

| | Polymorphism |
|---|---|
| Nucleotide location | (alternative nucleotides) |
| −476 | G/A |
| −364 | G/T |
| −339 | ΔG |
| −308 | T/G |
| −301 | T/G |
| −278 | T/G |
| −272 to −276 | CCAGA/SMRRR |
| −168 | T/C |
| −75 | A/G |
| −57 | G/T |
| −31 | ΔG |
| −6 | G/A |
| −1 | T/A/C |
| +3 | G/C |
| +16 | A/G |
| +26 | A/C |
| +59 | T/G |

The polymorphisms at positions −1, +3 and +59 are predicted to cause amino acid substitutions in the GHDTA protein, putatively encoded by this region of the GH1 gene promoter (see below). Some of the sequence variants occur in the same positions in which the GH1 gene differs from the other placentally-expressed genes suggesting that the mechanism might be gene conversion and that the placental genes have served as donors of the converted sequences.

In a study of prepubertal short children with GH insufficiency, Hasegawa et al [J. Clin. Endocrinol Metab 85 1290-1295 (2000)] reported an association between three polymorphisms in the GH1 gene [IVS4 C→T 1101, T/G −278 and T/G −57] and both GH secretion and height.

Since the first GH1 gene deletions were reported, a variety of more subtle lesions have been described. In some cases, these lesions have been associated with unusual types of GH deficiency and are potentially important as a means of obtaining new insights into GH structure and function

The gene encoding growth hormone (GH1) was one of the first human genes to be cloned and the first gross gene deletions (6.7 kb type) responsible for inherited growth hormone deficiency were soon detected by Southern blotting. All gross deletions involving the GH1 gene result in severe (type IA) deficiency, characterised by the total absence of GH. About 70% of characterised deletions of the GH1 gene are 6.7 kb in length, whilst most of the remainder are of 7.6 kb or 7.0 kb (Table 2B—Gross deletions involving the GH1 gene, or in the vicinity of the GH1 gene, that cause GH deficiency and short stature).

TABLE 2B
Gross deletions involving or in the vicinity of the GH1 gene

| | | | Post- |
|---|---|---|---|
| | | | treatment |
| Deletion | Loci | | antibodies |
| size (kb) | involved | Comments | present? |
| 6.7 | GH1 | Swiss family | Yes |
| 6.7 | GH1 | Japanese family | Yes |
| 6.7 | GH1 | Argentinan family of | Yes |
| | | Spanish ancestry. | |
| | | Homozygous. | |
| 6.7 | GH1 | Austrian family | Yes |
| 6.7 | GH1 | Brazilian family | Yes |
| 6.7 | GH1 | Patient with short stature | Yes |
| | | and cystic fibrosis | |
| 6.7 | GH1 | Various | No |
| 7.6 | GH1 | Iraqi, Yemeni and Iranian | No |
| | | families | |
| 7.6 | GH1 | Italian family. Homozygous. | Yes |
| | | Consanguinous marriage | |
| 7.6 | GH1 | Italian and Turkish families | Yes |
| 7.6 | GH1 | Spanish family | No |
| 7.6 | GH1 | Various | Yes |
| 7.0 | GH1 | Canadian family | Yes |
| 7.0 | GH1 | Mexican family | Yes |
| 7.0 | GH1 | Chinese family. | no - No |
| | | Homozygous | treatment |
| | | | with GH. |
| 45 | GH1, CSHP1, | Turkish family. | Yes |
| | CSH1, GH2 | Homozygous. | |
| | | Consanguinous marriage | |
| 45 | GH1, CSHP1, | Italian family. Homozygous | Yes |
| | CSH1, GH2 | | |
| 45 | GH1, CSHP1, | Italian family. Homozygous. | Yes |
| | CSH1, GH2 | Consanguinous marriage | |
| 45 | GH1, CSHP1, | “Asian” family | No |
| | CSH1, GH2 | | |
| ? | CSH1, GH2, | Italian family. Heterozygous | No |
| | CSH2 | | |
| ? | CSH1, GH2, | Danish family. Compound | No |
| | CSH2 | heterozygous for non- | |
| | | identical deletions | |
| Double | (i) GH1 | French origin (Romany). | Yes |
| | (6.7 kb) | Homozygous. | |
| | (ii) CSH1, | Consanguinous marriage. | |
| | GH2, CSH2 | | |
| | (˜32 kb) | | |

In addition, several examples of much more infrequent deletions have been reported. In recent years, various attempts have been made to move away from Southern blotting toward PCR-based approaches as a mutation screening tool. Homozygous GH1 gene deletions have been fairly readily detected by PCR amplification of the GH1 gene and flanking regions followed by restriction enzyme digestion of the resulting PCR products. Although this approach has been used successfully to exclude homozygosity for a GH1 gene deletion in at-risk pregnancies, it is however unable to distinguish homozygosity for the wild-type gene from heterozygosity for a gene deletion. It would also fail to detect deletions other than the relatively short 6.7, 7.0 and 7.6 kb deletions that remove only the GH1 gene.

PCR primers have been designed which immediately flank the GH1 gene and which generate a 790 bp fragment from control DNA samples. Absence of this fragment was held to be indicative of a GH1 gene deletion but the use of “non-specific PCR fragments” as internal controls for PCR amplification must make the reliability of this method somewhat suspect.

As well as gross deletions, three micro-deletions of the GH1 gene have been reported; two of these patients were also heterozygous for the 6.7 kb GH1 gene deletion (Table 3).

TABLE 3
Micro-deletions in the GH1 gene causing GH deficiency and short stature

| | Deletion | Codon | Post- |
|---|---|---|---|
| | (Lower case letters denote the deleted | (Numbering is relative to | treatment |
| Deficiency | bases. \hat{ } specifies the location of the | translational initiation codon | antibodies |
| type | numbered codon immediately downstream.) | ATG at −26.) | present? |
| IA | GCCTG\hat{ }CTCTGcCTGCCCTGGC | −11 | Yes |
| II | CCCCAGGCGGggatgggggagacctgtaGTCAGAGCCC | Intron 3 (del +28 | No |
| | | to +45) | |
| IA | TCTGT\hat{ }TTCTCagAGTCTATTCC | 54 | No |

Only seven different single base-pair substitutions have been reported from within the coding region of the GH1 gene (Table 4).

TABLE 4
Single base-pair substitutions in the GH1 coding
region causing GH deficiency and short stature

| | | | Codon | |
|---|---|---|---|---|
| | | | (numbering | |
| | | | relative to | |
| | | | translational | Post-treatment |
| Deficiency | Nucleotide | Amino acid | initiation codon | antibodies |
| type | substitution | substitution | ATG at −26) | present? |
| IA | ACA→GCA | Thr→Ala | −24 | No |
| IA | TGG→TAG | Trp→Term | −7 | No |
| IA | GAG→TAG | Glu→Term | −4 | Yes |
| II | CGC→TGC | Arg→Cys | 77 | No |
| ? | CCC→CTC | Pro→Leu | 89 | No |
| ? | GAC→GGC | Asp→Gly | 112 | No |
| ? | CGC→CAC | Arg→His | 183 | No |

Two of these single base-pair substitutions are nonsense mutations converting amino acid residues Trp-7 and Glu-4 in the signal peptide to stop codons. These mutations are the only known GH1 gene lesions to cause type IA deficiency that are not gene deletions. Since these lesions predict termination of translation within the signal peptide, they would be incompatible with the production of a functional GH molecule. The other five single base-pair substitutions (including R→C at codon 77, disclosed in EPA 790 305 in relation to the treatment of gigantism) are missense mutations that result in the production of dysfunctional growth hormone molecules. Such naturally-occurring mutations are very much more informative than artificially-induced mutations, in that the former can, in principle, be related directly to the clinical phenotype ie the height of the patient in question.

Single base-pair substitutions in the promoter region of possible pathological significance were first sought by sequencing the promoter region of the GH1 gene (between −60 and +70 relative to the transcriptional initiation site) in three Chinese patients with IGHD IA and 2 controls. Several differences were noted but these were probable polymorphisms and were not characterised further. As mentioned above, the promoter region of the GH1 gene has subsequently been shown to exhibit a very high level of sequence polymorphism with 17 variant nucleotides within a 570 bp stretch (FIG. 3). However, these sequence variants were not found to be over-represented in patients as compared to controls.

GH1 promoter variation has also been separately investigated and a total of 22 variant polymorphic sites were detected, mostly single base-pair substitutions: 17 of these occurred in a 550 bp region 5′ to the ATG initiation codon, three occurred around position −1075 5′ to ATG, and two occurred within intron 1 (IVS1) at positions 76 and 219 respectively [Wagner et al, Eur J Endocrinol 137 474-81 (1997)]. All except four of these variants were also noted in controls but these four variants were not considered to be the cause of the growth hormone deficiency. Only one of the variant sites occurred within a sequence homologous to a transcription factor binding site: the alternative presence of CCAGA and GAGAG sequences at −333 within a potential (but not proven) NF-1 binding site.

Therefore, to date, no mutations of pathological significance have been reported in the GH1 gene promoter.

Single base-pair substitutions affecting mRNA splicing have also been described in the GH1 gene. Most are associated with a comparatively rare dominant form of GH deficiency (Table 5).

TABLE 5
Single base-pair substitutions affecting mRNA splicing
and causing GH deficiency and short stature

| | Nucleotide | | |
|---|---|---|---|
| Deficiency | substitution/ | | Ethno-geographic |
| type | position | Splice site | origin/zygosity |
| II | G→A, +1 | IVS3 donor | Sweden, North America, |
| | | | Northern Europe, South |
| | | | Africa, Chile/heterozygous |
| II | G→C, +1 | IVS3 donor | Turkish/heterozygous |
| II | T→C, +2 | IVS3 donor | Russian/heterozygous |
| II | G→A, +5 | IVS3 donor | Chilean/heterozygous |
| II | G→C, +5 | IVS3 donor | Japanese/heterozygous |
| II | T→C, +6 | IVS3 donor | Turkish/heterozygous |
| | | | Asian/heterozygous |
| II | G→A, +28 | IVS3 donor | ?/heterozygous |
| IB | G→C, +1 | IVS4 donor | Saudi Arabian/homozygous |
| IB | G→T, +1 | IVS4 donor | Saudi Arabian/homozygous |
| IB | G→C, +5 | IVS4 donor | Bedouin/heterozygous |

The transversions in the intron 4 donor splice site have been shown by mRNA in vitro expression analysis of transfected cells to activate a cryptic splice site within exon 4, 73 bp 5′ to the exon 4 donor splice site. This would predict the generation of an aberrantly spliced product lacking amino acids 103-126 encoded by exon 4 and, as a consequence of a shift in the reading frame, the incorporation of 94 novel amino acids including 29 resulting from read-through of the normally untranslated 3′ non-coding region of the GH1 gene.

Since the region of the GH protein encoded by exons 4 and 5 is thought to be important for correct targeting of the protein to secretory granules, it has been predicted that this aberrant protein would not be secreted normally. However, no antibodies to exogenous GH have been noted in patients with type IB GH deficiency. The avoidance of immune intolerance may thus indicate that at least some of the aberrant protein product could be secreted and that it could be partially stable in the circulation. The seven known splicing mutations within IVS3 (Table 5) are associated with a type II deficiency state manifesting autosomal dominant inheritance through the affected families.

GH deficiency patients with truncating GH1 mutations or homozygous gene deletions are at considerable risk of developing anti-GH antibodies upon GH treatment. By contrast, we are not aware of any reports describing allo-antibody formation in patients with either missense mutations or single base-pair substitutions within splice sites.

Until now, no other correlations between mutant genotype and clinical phenotype have been reported. The requisite data in the published literature are sparse and very variable in quality, but we have attempted a crude meta-analysis as a means of gauging whether or not patients with gross gene deletions differ from patients with splice site mutations in terms of their clinical and phenotypic sequelae. The height of the patients with GH1 deletions was found to be on average 7.3 SD below the age-adjusted mean (n=29), as compared with an average of 5.4 SD below the mean (n=17) for the patients with GH1 splicing mutations. Although bone age delay was greater and growth velocity lower in the deletion patients, such findings are very difficult to interpret since they may be subject to bias of ascertainment.

Since most cases of familial GH deficiency hitherto described are inherited as an autosomal recessive trait, some examples of the inherited deficiency state are likely to have gone unrecognized owing to small family size. Similarly, cases of GH deficiency resulting from de novo mutations of the GH1 gene could be classified as sporadic, and a genetic explanation for the disorder would neither be entertained nor sought. Finally, depending upon the criteria used for defining the deficiency state, it may be that the full breadth of both the phenotypic and genotypic spectrum of GH deficiency may never have come to clinical attention. For these reasons, current estimates of the prevalence of GH deficiency could be inaccurate and may therefore seriously underestimate the true prevalence in the population.

The definition of IGHD favoured by many combines (a) severe growth retardation, often—as mentioned above—defined as <−4.5 SD in height; (b) reduced GH response to stimulation/provocation (ie a serum GH level of <4 ng/ml); and (c) no other cause for growth retardation. The strict adherence to formal definitions of what constitutes GH deficiency and the fairly uniform acceptance of these criteria, especially criterion (b), in selecting patients for study [Shalet S M et al. Endocrine Rev 19 203-223 (1998)] would have served to ensure that the described GH1 mutational spectrum was not only far from complete but also unrepresentative of the wider mutational spectrum.

We have proposed that moderating the criteria applied in selecting patients for study would be likely to lead to the inclusion of patients whose growth failure is a manifestation of a different portion of the GH deficiency spectrum, and which could therefore yield a novel set of underlying mutational lesions. Some of these novel lesions could give rise to stable, yet dysfunctional, GH molecules that would exhibit normal immunological reactivity but little or no biological activity. On the basis of radio-immunoassay test results, dysfunctional GH molecules would have been erroneously regarded as normal. If such dysfunctional variants were to turn out to be common, then it would follow that GH deficiency is being under-diagnosed as a result of our current dependence on radio-immunoassay-based GH “function tests”. Further, it would demonstrate an urgent need for the development of a true functional diagnostic assay.

We have therefore investigated a variety of patient cohorts and surprisingly found new variants of GH1 together with some corresponding GH protein variants encoded thereby.

Accordingly, the present invention provides a variant of GH1, selected from the group consisting of:
(a)
   (i) +480 C→T;
   (ii) +446 C→T;
   (iii) +1491 C→G;
   (iv) −60 G→A;
   (v) −40 to −39 GG→CT;
   (vi) −360 A→G; and
   (vii) +748 A→G
      (where figures relate to GH1 nucleotide position number, counting from TSS);
(b) a sequence substantially homologous to or that hybridises to sequence (a) under stringent conditions;
(c) a sequence substantially homologous to or that hybridises to the sequences (a) or (b) but for degeneracy of the genetic code; and
(d) an oligonucleotide specific for any of the sequences (a), (b) or (c) above.

By “substantially homologous” herein is meant that the nucleic acid sequence has at least 80% identity of its nucleotide bases with those of sequence (a), in matching positions in the sequence, provided that up to six bases may be omitted or added therein and further provided that the specified mutation is conserved. Preferably, the sequence has at least 90% homology and more preferred are sequences having at least 95% homology with the sequence (a). Such homologous sequences encode a protein having substantially the same biological activity, including functional activity, as the corresponding proteins encoded by the nucleic acid sequence variations of the invention.

Oligonucleotides “specific for” any of these nucleic acid sequences (a) to (c) above are useful for identifying and isolating the sequences of this invention, and comprise a unique sequence encoding a unique fragment of the amino acid sequence of the corresponding peptide.

Preferred variants according to (a) above are:
(a)
   (i) +480C→T; and
   (ii) +446C→T.

In particular, the present invention provides a nucleic acid sequence as defined above, wherein the sequence is a DNA or RNA sequence, such as cDNA or mRNA.

The present invention therefore also provides a transcript of a variant of GH1, such as a protein (hereinafter ‘GH variant’) comprising an amino acid sequence encoded by a variant of GH1, wherein the variant of GH1 is one according to this invention.

Accordingly, the present invention provides a GH variant, with reference to hGH, selected from:
(i) Thr27Ile, eg being encoded by the variant of GH1(a)(i) as defined above (namely, +480 C→T);
(ii) Arg16Cys, eg being encoded by the variant of CH1(a)(ii) as defined above (namely, +446C→T);
(iii) Ile179Met, being encoded by the variant of GH1(a)(i) as defined above (namely, +1491 C→T);
(iv) Asn47Asp, being encoded by the variant of GH1(a)(vi) as defined above (namely, +748 A→G).

Preferred variants of GH1 above are:
(i) Thr27Ile, eg being encoded by the variant of GH1(a)(i) as defined above (namely, +480 C→T);
(ii) Arg16Cys, eg being encoded by the variant of GH1(a)(ii) as defined above (namely, +446C→T); and
(iii) Ile179Met, being encoded by the variant of GH1(a)(i) as defined above (namely, +1491 C→G).

Especially preferred variants of GH1 above are:
(i) Thr27Ile, eg being encoded by the variant of GH1(a)(i) as defined above (namely, +480 C→T); and
(ii) Arg16Cys, eg being encoded by the variant of GH1(a)(ii) as defined above (namely, +446C→T).

The above-identified variants of GH1 or protein encoded thereby can give rise to the following advantages:
1. Expansion of the known spectrum of GH1 gene mutations by identification and characterisation of new lesions.
2. Evaluation of the role of GH1 gene mutations in the actiology of short stature.
3. Identification of the mode of inheritance of novel GH1 gene lesions.
4. Elucidation of the relationship between mutant genotype and clinical phenotype. This is deemed essential for the early detection and appropriate clinical management of GH deficiency.
5. Evaluation of the effects of GH1 mutations on the structure and function of the GH molecule. This is particularly important for the assessment of those children with a clinical phenotype at the milder end of the clinical spectrum of short stature. In this group of patients, dysfunctional GH may be produced that is immunologically active and therefore falls within the normal range in GH function tests.
6. Development of rapid DNA diagnostic tests for inherited GH deficiency

Therefore, the characterisation of further, naturally occurring GH1 lesions promises to be of considerable importance to studies of GH structure, function and expression. Studies of novel coding sequence variants should increase our understanding not only of GH function, but also of the interactions between GH and its receptor (GHR), and the process of GHR-mediated signal transduction. Insights obtained could be relevant to the rational design of a new generation of therapeutic agents. Similarly, studies of naturally-occurring GH1 lesions in the promoter region should provide new insights into the control of GH1 gene expression. Thus it may be seen that a broad spectrum of mutational lesions will necessarily improve our understanding of the relationship between mutant genotype and clinical phenotype in inherited forms of GH deficiency. Clearly, these studies are essential for the early detection and appropriate clinical management of familial GH deficiency.

Accordingly, the present invention provides a screening method for screening a patient suspected of having dysfunctional GH, which screening method comprises the steps of:
(a) obtaining a test sample comprising a nucleotide sequence of the human GH1 gene from the patient; and
(b) comparing a region of the sequence obtained from the test sample with the corresponding region of a predetermined sequence characterised in that the predetermined sequence is selected from a variant of GH1 of the present invention.

More specifically, the screening method of the invention is characterised in that the predetermined sequence is an oligonucleotide having a nucleic acid sequence corresponding to a region of a variant GH1 gene, which region incorporates at least one variation selected from those defined herein, when compared with the corresponding region of the wild type sequence.

Preferably, the test sample comprises genomic DNA, which may be extracted by conventional methods.

Conveniently, the present invention provides a screening method for screening an individual suspected of GH dysfunction, which screening method comprises the steps of:
(a) obtaining a test sample comprising a nucleotide sequence of the human GH1 gene from an individual; and
(b) comparing a region of the sequence obtained from the test sample with the corresponding region of a predetermined sequence
   wherein the predetermined sequence is selected from a GH1 variant according to this invention.

The predetermined sequence is preferably an oligonucleotide having a nucleic acid sequence corresponding to a region of a variant GH1 gene according to this invention, which region incorporates at least one variation when compared with the corresponding region of the wild type sequence.

The first test sample or the test sample in the screening methods of this invention preferably comprises genomic DNA.

In the screening method of the invention, the comparison step may be carried out in conventional manner, for example by sequencing the appropriate region of the GH1 gene, particularly in the case where relatively few variants are to be detected/compared. Where relatively large numbers of variants are involved, DNA chip technology may be employed, such as wherein the chip is a miniature parallel analytical device that is used to screen simultaneously either for multiple known mutations or for all possible mutations, by hybridisation of labelled sample DNA (cDNA or genomic DNA derived from the patient) to micro-arrays of mutation-specific oligonucleotide probes immobilised on a solid support [Southern, Trends Genet 12 110-115 (1996)].

The advantage of a DNA screening method according to the invention over current tests include:
1. It involves, for the patient, only a single blood test that can be performed in a clinic. Hospital admission, prolonged medical supervision and repeated blood sampling would not be required as is the case for the majority of currently-available tests. There would therefore be a reduction in the expense incurred, the use of specialist time and the distress caused for each patient tested.
2. Earlier diagnosis of functional GH deficiency in patients would become possible. The ease with which the DNA screen can be performed would allow the clinician to consider such an investigation much earlier in the management of a patient than might otherwise be the case. Currently, owing to the problems inherent in tests for GH secretion, doctors will assess children in the out-patient clinic over a long period of time, sometimes several years, before they will subject a child to an IST. The early diagnosis of a genetic aetiology for GH deficiency would enable earlier treatment with GH thereby bringing forward the opportunity to treat patients appropriately by months, or even years in individuals with a less severe phenotype.
3. More patients could be tested for GH dysfunction. The ease of the DNA test would allow the doctor to perform it as part of the initial assessment of all short patients at their first visit to the endocrine clinic. This is likely to reveal patients with lesions of the GH1 gene that cause severe growth problems and also those with milder lesions (e.g. missense mutations in the coding region). These patients may not previously have come to clinical attention because their clinical/phenotypic problems would not have been severe enough to warrant an IST, but they might nevertheless still benefit from treatment with GH.
4. Early identification of patients who will require life-long treatment with GH would be possible. These patients could be identified and treated appropriately without recourse to either initial testing or re-testing for GH secretion, or the use of a period without GH to assess their progress (a “trial without treatment”).
5. Easy and early identification of family members with GH dysfunction would become available. Once the genetic lesion responsible for growth problems has been identified in an individual, it is relatively easy to assess other family members for the same genetic lesion and to ascertain whether they would also gain benefit from treatment with GH.
6. Accuracy of diagnosis should increase. Tests for GH secretion are notorious for their variability in terms of reproducibility of assay results, both within and between laboratories. DNA screening would make this problem a thing of the past. In addition, GH secretion test results can be very difficult to interpret in certain situations, for example, if the patient is also hypothyroid or has delayed puberty. DNA screening would remove this doubt and prevent delay in the initiation of GH treatment for those patients in whom its use would be beneficial.

Accordingly, the present invention further provides a kit suitable for use in carrying out the screening method of the invention, which kit comprises:
(a) an oligonucleotide having a nucleic acid sequence corresponding to a region of a variant GH1 gene, which region incorporates at least one variation from the corresponding wild-type sequence selected from variations according to the present invention; and
(b) an oligonucleotide having a nucleic acid sequence corresponding to the wild-type sequence in the region specified in (a); and, optionally,
(c) one or more reagents suitable for carrying out PCR for amplifying desired regions of the patient's DNA.

Such reagents may include, for example, PCR primers corresponding to an exon of the GH1 gene, and/or primers defined herein; and/or other reagents for use in PCR, such as Taq DNA polymerase.

Preferably, the oligonucleotides in the kit comprise in the range of from 20 to 25 base-pairs, such as 20 base-pairs for the variant sequences and either 20 for the wild-type in the case where the variant is a single base-pair substitution or 25 base-pairs where the variant is a 5 base-pair deletion. In any case, the oligonucleotides must be selected so as to be unique for the region selected and not repeated elsewhere in the genome.

Obviously, in the situation where it is desired to screen for multiple variations, such as in the range of from 15 to 20 or more, this would necessitate a kit comprising up to 40 oligonucleotides or more. In the alternative screening method, therefore, using DNA chip technology, the present invention provides a plurality of oligonucleotides as defined in kit component (a) above immobilised on a solid support.

Other nucleotide detection methods could be used, such as signal amplification methods being pioneered in nanotechnology (such as Q-Dots). Also, single molecule detection methods could be employed (such as STM). In which case, the kit according to this invention may comprise one or more reagents for use in such alternative methods.

Alternatively, the screening method and corresponding kit according to this invention may be based on one or more so-called ‘surrogate markers’ that are indicative of or correlated to the presence of a variant of GH1 or a GH variant, such as proteins/amino acid sequences eg antibodies specific for a GH variant or a variant of GH1. Such a “surrogate marker” may comprise:
(a) any biomolecule (including, but not limited to, nucleotides, proteins, sugars, and lipids);
(b) a chemical compound (including, but not limited to, drugs, metabolites thereof, and other chemical compounds); and/or
(c) a physical characteristic,
   whose absence, presence, or quantity in an individual is measurable and correlated with the presence of a GH variant or a variant of GH1 according to the present invention.

Further, suitable, alternative screening methods according to this invention may further comprise obtaining a test sample comprising a GH variant (ie a protein/peptide sequence comprising a variation of hGH, such as one encoded by a variant of GH1 of this invention) that is identifiable by conventional protein sequence methods (including mass spectroscopy, micro-array analysis, pyrosequencing, etc), and/or antibody-based methods of detection (eg ELISA), and carrying out one or more such protein sequencing method(s).

In which alternative cases, the kit according to this invention may comprise one or more reagents for use in such alternative methods.

GH1 variants of this invention may have additional uses than as standards in a screening test for GH dysfunction. For example, variants other than those where the variation is in the promoter region of the GH1 gene may be used to treat a patient wherein GH production is over-stimulated, such as in cases of pituitary gigantism or acromegaly.

The present invention further provides:
(a) for the use of one or more of the GH variants or a variant of GH1 which comprises two terminating mutations for the identification of individuals who do not produce any growth hormone at all and who would be classified as classical GHD by conventional diagnostic techniques;
(b) a GH variant or a variant of GH1 which leads to modified binding of GH to the growth hormone receptor or its binding protein (ie the carrier for GH in vivo), insomuch as the transport of the variant GH from the pituitary by binding to its binding protein is impaired or inhibited leading to destruction of the unbound protein en route to the tissue receptor;
(c) a GH variant or a variant of GH1 capable of disrupting the formation of the zinc dimer storage form of the GH protein in the pituitary;
(d) a GH variant or a protein expressed by a variant of GH1, being a protein with antagonist properties to the GH receptor and whose receptor binding constant determines the amount of extraneous GH (dose) needed to treat a patient in order to overcome the potency and inhibitory action of the variant protein; ie the variant protein competes with the wild type to bind to the receptor;
(e) use of the GH variant or a variant of GH1 according to the invention for therapeutic, diagnostic or detection methods;
(f) use of the GH variant or a variant of GH1 according to the invention for the determination of susceptibility to a disease in an individual;
(g) use of the GH variant or a variant of GH1 according to the invention for the determination of susceptibility to a disease, including diabetes, obesity, infection, cancers or cardiac conditions;
(h) use of the GH variant or a variant of GH1 according to the invention for determining GH binding defects and/or pituitary storage defects;
(i) use of the GH variant or a variant of GH1 according to the invention for the determination of the diagnostic dose of antagonist treatment in acromegaly;
(j) use of the GH variant or a variant of GH1 according to the invention for use in medical treatment;
(k) use of the variant of GH1 according to the invention for use in gene therapy;
(l) use of the GH variant or a variant of GH1 according to the invention for determining one or more polymorphism(s) associated with a disease state; and
(m) use of the GH variant or a variant of GH1 according to the invention for the preparation of a therapeutic composition, diagnostics composition or kit, or detection kit for preventing, treating, diagnosing or detecting a condition associated with or caused by GH dysfunction in an individual.
(n) an oligonucleotide of about 20 nucleotides in length having a nucleic acid sequence corresponding to a region of a variant GH1 gene, which region incorporates at least one variation from the corresponding wild type sequence, said variation comprising one or more of those according to this invention;
(o) an oligonucleotide comprising the complement of the oligonucleotide of (n);
(p) an oligonucleotide of (n), wherein the nucleotide corresponding to the variation is located at the 3′ end of the molecule;
(q) a single-stranded DNA probe that hybridizes to a variant GH1 gene and not to a wild type GH1 gene, wherein the variant GH1 gene is selected from those according to this invention;
(r) an array of nucleic acid molecules attached to a solid support, the array comprising a single stranded DNA probe according to (q);
(s) a screening method for screening an individual suspected of GH dysfunction, which screening method comprises the steps of:
   (i) obtaining a test sample comprising a nucleotide sequence of the human GH1 gene from the individual; and
   (ii) comparing the sequence of a region of the human GH1 gene from the individual corresponding to a region of a variant GH1 gene according to (n);
(t) a method according to (s), wherein the comparing step involves hybridization with the predetermined sequence;
(u) a method according to (s), wherein the comparing step comprises amplifying at least a portion of a nucleic acid encoding human GH1;
(v) a method according to (s), wherein the comparing step comprises amplifying at least a portion of a nucleic acid encoding human GH1 with one or more oligonucleotide(s) selected from those described herein;
(w) an amplification oligonucleotide selected from those described herein;
(x) a diagnostic kit comprising the required components for the determination of the identity of one or more variations (including substitutions, insertions or deletions with respect to the wild type) of an individual's GH1 gene, as described herein, in particular a variation according to one or more of (n) to (q), above, and especially a diagnostic kit comprising an oligonucleotide for use in amplifying a segment of such a gene comprising a polymorphic site;
(y) an antibody specific for a variation as described herein from the reference hGH sequence and which antibody is capable of distinguishing between the variant and corresponding wild type amino acids at the indicated amino acid position; and
(z) a diagnostic kit comprising an antibody according to (y).

The present invention further provides a composition comprising a GH variant of this invention in association with a pharmaceutically acceptable carrier therefor.

Furthermore, the invention provides:
(a) a vector comprising a nucleic acid sequence according to the present invention;
(b) a host cell comprising the vector (a), such as a bacterial host cell; and
(c) a process for preparing a GH variant according to this invention, which process comprises:
   (i) culturing the host cell (b); and
   (ii) recovering from the culture medium the GH variant thereby produced.
(d) a protein or amino acid sequence being in culture medium and encoded or expressed by a sequence, vector, or cell as defined above.

The present invention will now be illustrated with reference to the following Examples.

### EXAMPLE 1A

### Patient Selection—UK Study

Sources of Patients

Children with short stature have been identified through referral to the Regional Paediatric Growth, Endocrine and Diabetes Service at the University of Wales College of Medicine in Cardiff and by collaboration with other similar UK centres (viz Newport, Birmingham, Bristol, Wrexham, Liverpool, Stoke-on-Trent, Portsmouth and Southampton). A full clinical history has been taken including family history, pedigree, documentation of growth parameters and previously-performed endocrine investigations. Accurate auxology was recorded wherever possible for the index case, parents and siblings. Blood samples for molecular genetic analysis were taken from the index case and appropriate close relatives. Further families were referred by Professor John A. Phillips III (Nashville, Tenn., USA), Dr Mohamad Maghnie (Pavia, Italy) and Dr Tamas Niederland (Gyor, Hungary). To date, samples from 83 GH-deficient families have been collected. Results relating to the first 70 patients are given in our co-pending patent specification no. PCT/GB01/2126.

Criteria Used

Criteria used for the selection of patients were:
(i) Growth below lower limit of % target height range, defined as a growth pattern [delineated by a series of height measurements; Brook C D G (Ed) Clinical Paediatric Endocrinology 3rd Ed, Chapter 9, p 141 (1995, Blackwell Science)] which, when plotted on a standard height chart [Tanner et al Arch Dis Child 45 755-762 (1970)], predicts an adult height for the individual which is outside the individual's estimated target adult height range, the estimate being based upon the heights of the individual's parents;
(ii) Height velocity <25^{th} centile [Tanner J M, Whitehouse R H Atlas of Children's Growth (1982, London: Academic Press)]; and Butler et al Ann Hum Biol 17 177-198 (1990) are sources for statistics enabling a determination of this criterion, viz that the height velocity of the patient is less than the 25^{th} centile for the patient's age];
(iii) Bone age delay of at least 2, for example, 3.5-4 years when compared with chronological age, except in children of 5 or fewer years of age or in those with clinical evidence of pubertal development [The Tanner-Whitehouse scale for assessing years of bone age delay is described by Tanner J M, Whitehouse R H, Cameron N et al in Assessment of Skeletal Maturity and Prediction of Adult Height (1983, London: Academic Press). Assessment of bone age delay in an individual is subject to a greater level of variation, when carried out more than once, the younger the individual, so, for example, multiple assessments of a child of age two may result in a bone age delay varying by +/−6 months, but at age 3 might vary by +/−4 months, and so on;
(iv) All other investigations normal; and
(v) Growth hormone secretion tests normal.

Criteria (iv) and (v) may be summarised as “no identifiable pathology, other than the possibility of a GH axis defect that could account for the observed growth failure. Furthermore, the key criterion for inclusion in this study was that the clinician assessing the child should have had sufficient concern with regard to the child's growth pattern to warrant GH secretion testing. The children selected exhibited a clinical phenotype that resulted in sufficient clinical concern to have warranted GH secretion testing, regardless of the type of test, the test results, or indeed whether the child attended for testing.

In Table 5B: *GH FT: peak: Signifies units (IU/L) of activity in one or more standard Growth Hormone Function Tests. ‘Random’ denotes GH measurement taken randomly. ND denotes ‘test not done’. The height centile is included to demonstrate that it is not an essential selection criterion to have a height substantially below the 50^{th} centile; we have found variations in GH/GH1 that occur even in patients not having a substantially reduced height.

Patients in italics produced samples exhibiting variations. Those also in bold exhibited novel variations.

TABLE 5B
Patients studied and results of criteria used

| | | Growth | Bone Age | GH FT: |
|---|---|---|---|---|
| Patient | Height | Velocity | Delay | peak |
| No. | Centile | Centile (ii) | (years) (iii) | (v) |
| 71 | 0.4 | <25 | | 1.3 |
| 72 | | <25 | | |
| 73 | | <25 | | |
| 74 | | <25 | | |
| 75 | <<0.4 | <25 | 0.5 at 14 | 6.8 at 13; |
| | | | | N at 19 |
| 76a | 0.4 | <25 | 0 at 10.5 | 18.3 |
| 76b | <0.4 | <25 | 1 at 8 | 16.4 |
| 77 | <0.4 | <25 | | |
| 78 | <0.4 | <25 | | |
| 79 | <0.4 | <25 | | |
| 80 | <0.4 | <25 | | |
| 81 | <0.4 | <25 | | |
| 82 | <0.4 | <25 | | |
| 83 | <0.4 | <25 | | Random |
| | | | | normal |
| 84 | <0.4 | <25 | | |
| 85 | <0.4 | <25 | | |
| 86 | <0.4 | <25 | | |

### EXAMPLE 1B

### Patient Selection—Andalucia Study

A different patient cohort was established in Andalucia, Spain. Fifty patients were selected on the basis of their classification as FSS, ie exhibiting familial short stature, as defined by Ranke in Hormone Research 45 (Suppl 2) 64-66 (1996). Such patients have at least one genetic family member exhibiting short stature.
Patient B53, Height: −2.0 SD
   mother's height 149.5 cm (−2.15 SD)
   father's height: 163.3 (−1.71 SD)
   peak GH test: 18.1 ng/ml (clonidine)
   IGF-I: 94 ng/ml
   IGFBP-3: 2.03 mg/L
   BD: 10/6/91
Patient B49, height: −2.7 SD
   mother's height 138.9 cm (−3.88 SD)
   father's height: 165.4 (−1.40 SD)
   peak GH test: 10.4 ng/ml (propanolol)
   IGF-I: 94 ng/ml
   IGFBP-3: 2.97 mg/L
   BD: 13/12/92
Patient B4, Height: −2.1 SD
   mother's height: 163.4 cm (−1.7 SD)
   father's height: 148.7 cm (−2.3 SD)
   IGF-I: 99 ng/ml
   IGFBP-3: 2.1 mg/L

### EXAMPLE 2

### Polymerase Chain Reaction (PCR) Amplification of a GH1-Specific Fragment

PCR amplification of a 3.2 kb GH1-specific fragment has been performed on the patients selected as per Example 1 and controls. Genomic DNA was extracted from patient lymphocytes by standard procedures.

Oligonucleotide primers GH1F (5′ GGGAGCCCCAGCAATGC 3′; −615 to −599) and GH1R (5′ TGTAGGAAGTCTGGGGTGC 3′; +2598 to +2616) were designed to correspond to GH1-specific sequences in order to PCR amplify a 3.2 kb single genomic DNA fragment containing the human GH1 gene using the Expand™ high fidelity system (Roche).

Two separate thin-walled 0.65 ml PCR tubes were used for each reaction. The first tube contained 500 nanograms (ng) each primer (GH1F and GH1R), 200 μM dATP, dTTP, dCTP and dGTP and 200 ng of patient genomic DNA made up to a final volume of 25 μl with sterile water. The second tube contained 5 μl 10× reaction buffer made up to a final volume of 24.25 μl with sterile water. Both tubes were placed on ice for 5 minutes. After this time, 0.75 μl of Expand™ polymerase mix was added to the second tube, the contents mixed and transferred to the first tube. The tube was centrifuged for 30 seconds and the reaction mixture overlaid with 30 μl light mineral oil (Sigma). The reaction mixture was then placed in a 480 or 9700 PCR programmable thermal cycler (Perkin Elmer) set at 95° C.

The reaction mix was then amplified under the following conditions: 95° C. for 2 minutes followed by 30 cycles of 95° C. for 30 seconds, 58° C. for 30 seconds and 68° C. for 2 minutes. For the last 20 cycles, the elongation step at 68° C. was increased by 5 seconds per cycle. This was followed by a further incubation at 68° C. for 7 minutes and the reaction was then cooled to 4° C. prior to further analysis. For each set of reactions, a blank (negative control) was also set up. The blank reaction contained all reagents apart from genomic DNA and was used to ensure that none of the reagents were contaminated.

A one-tenth volume (5 μl) was analysed on a 1.5% agarose gel to assess whether PCR amplification had been successful before nested PCR was performed. Those samples that had PCR-amplified successfully were then diluted 1 in 100 prior to use for nested PCR.

### EXAMPLE 3

### Nested-PCR

Nested PCR was performed on the fragments produced in Example 2 to generate, in each case, seven overlapping sub-fragments that together span the entire GH1 gene. In addition, the Locus Control Region has been PCR-amplified (see Example 5) in all but three patients.

The seven overlapping sub-fragments of the initial 3.2 kb PCR product were PCR-amplified using Taq Gold DNA polymerase (Perkin-Elmer). Oligonucleotides used for these reactions are listed in Table 6 together with their sequence locations as determined from the GH1 gene reference sequence.

A 1 μl aliquot of the diluted long (3.2 kb) PCR product was put into a thin-walled 0.2 ml PCR tube or into one well of a 96-well microtitre plate. To this was added 5 μl 10× reaction buffer, 500 ng appropriate primer pair (e.g. GH1DF and GH1DR), dATP, dTTP, dCTP and dGTP to a final concentration of 200 μM, sterile water to a volume of 49.8 μl, followed by 0.2 μl Taq Gold polymerase.

The tube or microtitre plate was then placed in a Primus 96 thermal cycler (MWG Biotech) and cycled as follows: 12 min 95° C. followed by 32 cycles of 95° C. for 30 seconds, 58° C. for 30 seconds and 72° C. for 2 minutes. This was followed by further incubation at 72° C. for 10 minutes and the reaction was then cooled to 4° C. prior to further analysis.

A one-tenth volume (5 μl) of the reaction mix was analysed on a 0.8% agarose gel to determine that the reaction had worked before denaturing high-pressure liquid chromatography (DHPLC) was performed on a WAVE™ DNA fragment analysis system (Transgenomic Inc. Crewe, Cheshire, UK). To enhance heteroduplex formation, the PCR product was denatured at 95° C. for 5 minutes, followed by gradual re-annealing to 50° C. over 45 minutes. Products were loaded on a DNAsep column (Transgenomic Inc.) and eluted with a linear acetonitrile (BDH Merck) gradient of 2%/min in a 0.1M triethylamine acetate buffer (TEAA pH 7.0), at a constant flow rate of 0.9 ml/minute. The start and end points of the gradient were adjusted according to the size of the PCR product. Analysis took 6.5-8.5 minutes per amplified sample, including the time required for column regeneration and equilibration. Samples were analysed at the Melt temperatures (TM) determined using the DHPLCMelt software (http://insertion.stanford.edu/melt.html) and listed in Table 6. Eluted DNA fragments were detected by an UV-C detector (Transgenomic Inc.).

TABLE 6
Oligonucleotide primers used for DHPLC analysis and DNA sequencing

| | | | | DHPLC melt |
|---|---|---|---|---|
| Fragment | Primer | Sequence (5′ to 3′) | Position | temperature |
| 1 | GH1DF | CTCCGCGTTCAGGTTGGC | −309 to −292 | 60° C. |
| | GH1DT | CTTGGGATCCTTGAGCTGG | −8 to +11 | |
| 2 | GH2DF | GGGCAACAGTGGGAGAGAAG | −59 to −40 | 63° C. |
| | GH2DR | CCTCCAGGGACCAGGAGC | +222 to +239 | |
| 3 | GH3DF | CATGTAAGCCCAGTATTTGGCC | +189 to +210 | 62° C. |
| | GH3DR | CTGAGCTCCTTAGTCTCCTCCTCT | +563 to +586 | |
| 4 | GH4DF | GACTTTCCCCCGCTGGGAAA | +541 to +560 | 62° C. |
| | GH4DR | GGAGAAGGCATCCACTCACGG | +821 to +841 | |
| 5 | GH5DF | TCAGAGTCTATTCCGACACCC | +772 to +792 | 62° C. |
| | GH5DR | GTGTTTCTCTAACACAGCTCTC | +1127 to +1148 | |
| 6 | GH6DG | TCCCCAATCCTGGAGCCCCACTGA | +1099 to +1122 | 62° C. |
| | GH6DR | CGTAGTTCTTGAGTAGTGCGTCATCG | +1410 to +1435 | |
| 7 | GH7DF | TTCAAGCAGACCTACAGCAAGTTCG | +1369 to +1393 | 57° C. and 62° C. |
| | GH7DR | CTTGGTTCCCGAATAGACCCCG | +1731 to +1752 | |

With respect to the samples obtained from patients selected according to Example 1A above, the following procedures (Examples 4 & 5) were carried out:

### EXAMPLE 4

### DNA-Sequencing of GH1-Specific Long PCR Fragments

Clones containing the GH1-specific long PCR fragment were sequenced with the BigDye (RTM) sequencing kit (Perkin Elmer) in either 0.2 ml tubes or 96-well microtitre plates in a Primus 96 (MWG) or 9700 (Perkin Elmer) PCR thermal cycler. Oligonucleotide primers used for sequencing were:

GH1S1
(5′ GTGGTCAGTGTTGGAACTGC 3′: −556 to −537);
GH3DF
(5′ CATGTAAGCCAAGTATTTGGCC 3′: +189 to +210);
GH4DF
(5′ GACTTTCCCCCGCTGTAAATAAG 3′: +541 to +560):
and
GH6DF
(5′ TCCCCAATCCTGGAGCCCCACTGA 3′: +1099 to +1122).

1 μg of cloned DNA was sequenced with 3.2 pmol of the appropriate primer and 4 μl BigDye sequencing mix in a final volume of 20 μl. The tube or microtitre plate was then placed in the thermal cycler and cycled as follows: 2 minutes 96° C. followed by 30 cycles of 96° C. for 30 seconds, 50° C. for 15 seconds and 60° C. for 4 minutes. The reaction was then cooled to 4° C. prior to purification.

Purification was performed by adding 80 μl 75% isopropanol to the completed sequencing reaction. This was then mixed and left at room temperature for 30 minutes. The reaction was then centrifuged at 14,000 rpm for 20 minutes at room temperature. The supernatant was then removed and 250 μl 75% isopropanol was added to the precipitate. The sample was mixed and centrifuged for 5 minutes at 14,000 rpm at room temperature. The supernatant was removed and the pellet dried at 75° C. for 2 minutes.

Samples were then analysed on an ABI Prism 377 or 3100 DNA sequencer.

### EXAMPLE 5

### GH1 Gene Mutations and Polymorphisms

(a) Example 1A (UK) Patients

Two new mutations have been found in our patient cohort in the GH1 gene promoter region: a single base-pair substitution and an indel.
(i) The −60 G→A substitution was found in 2/25 mutant alleles in our patient sample (in heterozygous form in patients 57 and 75). This mutation occurred within a stretch of five Gs in the vitamin D response element (Alonso et al Biochem Biophys Res Commun 247: 882-887 (1998)) in a base that is conserved in all mammals. This is consistent with the functional importance of this nucleotide. This variation is always associated with promoter haplotype 19, which is a low expressor.

(ii) The −40 to −39 GG→CT indel in patient 76A could have been templated by gene conversion (the donor sequence of the former being GH2, CSH2 or CSHP1 and the latter, CSH1 or CSH2).

TABLE 2
New GH1 gene mutations found in patients with GH deficiency

| | | Confirmed | Confirmed | Family |
|---|---|---|---|---|
| Patient | Mutation | (cloning) | (PCR/sequencing) | studies |
| 57 | −60 G → A | Yes | Yes | Maternal |
| 75 | −60 G → A | Yes | Yes | TBC |
| 76A | −216 A→G | Yes | No | Not in parents. |
| | −40 to −39 | | | De novo? |
| | GG→CT | | | |

(b) Example 1B (Barcelona) Patients

Three mutations of potential pathological importance were found in the sequence analysis of the 50 familial short stature patients from Barcelona: −360 A→G (Patient B4), GTC→ATC at +1029 (Val 110→Ile) (Patient B53; this variation is also described in co-pending patent specification no. PCT/GB01/2126) and ATC→ATG at +1491 (Ile179→Met) (Patient 49).

Since four Ile110 alleles were noted in the control sample (a frequency of 0.025), this variant occurs at polymorphic frequencies in the general population. Molecular modelling suggested that this substitution might exert a deleterious effect on the structure of GH; the evolutionarily-conserved Val110 residue forms part of the hydrophobic core at the N-terminal end of helix 3, and its replacement by Ile with its longer sidechain would be expected to cause steric hindrance. Consistent with this prediction, the Ile110 variant is associated with a dramatically reduced ability (40% of normal) to activate the JAK/STAT signal transduction pathway. The Val110→Ile substitution appears therefore to represent a functional polymorphism that is associated with a reduction in GH activity and which is potentially able to influence stature. This variation is associated with promoter haplotype 2, which has fairly normal activity.

With respect to the Ile179Met variation: Ile179 is positioned at the surface of the hGH protein centrally in helix 4. In the hGHbp/hGH 2:1 complex, Ile179 interacts directly with the ‘hot-spot’ residues of site 1, TRP104 and TRP169. It is therefore likely that a substitution of Ile179 with a methionine residue would interfere with a precise steric constraint in site 1, resulting in a significant change in the functioning of the hGH.

(c) Studies of GH1 Coding Sequence Variation in Controls

A total of 80 healthy British controls of Caucasian origin were also screened for variants, using the method of Examples 2 and 3, within the coding region of the GH1 gene. Five examples of silent substitutions found in single individuals were noted [GAC→GAT at Asp26, TCG→TCC at Ser85, TCG→TCA at Ser85, ACG→ACA at Thr123 and AAC→AAT at Asn109]. The Thr123 polymorphic variant has been reported previously (Counts et al Endocr Genet 2 55-60 (2001)).

In addition, three missense substitutions were noted [ACC→ATC, Thr27→Ile; AAC→GAC, Asn47→Asp; GTC→ATC, Val110→Ile, 1, 1 and 4 alleles respectively/160 alleles]; only the Val110→Ile substitution had been found in the patient study disclosed in our co-pending patent specification no. PCT/GB01/2126 (patient 66). Molecular modelling suggested that this substitution might exert a deleterious effect on the structure of GH; Val110 forms part of the hydrophobic core at the N-terminal end of helix 3 and its replacement by Ile with its longer sidechain would cause steric hindrance. It may thus be that while the Val110→Ile substitution in both control and patient populations, it is nevertheless capable of influencing stature. Other comments apply as in Example 5(b) above. This notwithstanding, the relative paucity of missense mutations in the control population argues in favour of the pathological significance of the lesions found in the patient cohort.

(d) Additional Results

In addition to the promoter haplotype associations mentioned with respect to Val110→Ile and −60G→A above, it has been found that the −24Thr→Ala (see Table 4, above) is always associated with promoter haplotype 21, which is a low expresser; and −48G→A (described in our co-pending patent specification no. PCT/GB01/2126) is always associated with promoter haplotype 2, which is a normal expresser.

### EXAMPLE 6

### Further Studies, Including Identification of Arg16Cys Variation

In Vitro Expression and Assay of Biological Activity of GH Variants

A cloned wild-type GH1 cDNA was PCR amplified using primers GHCDNA5 (5′ AAGCTTGCAATGGCTACAGGCTCCC 3′; −3 to +16) and GHCDNA3 (5′ ACCGGTCTAGAAGCCACAGCTCCC 3′; +636 to +654) where non-templated restriction sites for HindIII and AgeI are underlined. This PCR fragment was digested with HindIII and AgeI, cloned into the insect expression vector pIZ/V5-His (Invitrogen), and sequence-checked.

Site-directed mutagenesis was performed on wild-type GH1 cDNA using the QuikChange site-directed mutagenesis kit (Stratagene) according to the manufacturer's instructions. The vector was then transfected using Cellfectin into High Five insect cells (Invitrogen) grown in Express Five SFM medium (Invitrogen). Stably transfected cells were selected on the basis of their zeocin resistance. Medium was harvested when cells had grown to 80% confluence for two successive 7-day periods. Human GH in the culture supernatants was quantified by IRMA (Nichols Institute Diagnostics). With the exception of the Arg16Cys variant (which showed no cross-reactivity in the IRMA), the cross-reactivity of the GH variants and insect cell-expressed wild-type GH in the IRMA was confirmed to be equal to that of the assay reference preparation (calibrated against the National Institutes of Health's reference preparation NIAMDD-hGH-RP-1) by dilutional analysis.

The Arg16Cys variant was quantified by Western blotting, by comparing the intensity of the variant band with those produced by known quantities of wild-type GH. 10 μl culture medium from insect cells expressing the Arg16Cys variant were run on a 12% polyacrylamide gel together with varying amounts of wild-type GH (7-53 ng). The gel was electroblotted onto PVDF membrane as described (Lewis et al., 2002), probed with an anti-human GH antibody (Lab Vision), diluted 1:500 and visualised by enhanced chemiluminescence (ECL Plus, Amersham Pharmacia Biotech). Films were analysed by imaging densitometry and a standard curve constructed for wild-type GH. This curve was then used for quantification of the Arg16Cys variant (average of 6 separate measurements). IRMA quantification was confirmed by Western blotting. Equal quantities of variant and wild-type GH were loaded and the intensity and molecular weight (22 kD) of variant and wild-type bands were found to be indistinguishable in all cases.

HK293 cells, transfected with the full-length human GH receptor (GHR) and selected on the basis of elevated GHR expression (HK293Hi cells), were used to assay the biological activity of the GH variants (Ross et al Mol Endocrinol 11 265-73 (1997), von Laue et al J Endocrinol 165 301-11 (2000)). Cells were plated into 24-well plates (100,000 cells per well) for 24 hrs in DMEM:F-12 (1:1) containing 10% FCS. Cells were co-transfected overnight using a lipid-based transfection reagent (FuGENE 6, Roche Molecular Biochemicals) with a STAT5-responsive luciferase reporter gene construct (Ross et al, ibid) and a constitutively expressed β-galactosidase expression vector (pCH110; Amersham Pharmacia Biotech) to correct for transfection efficiency. Cells were then incubated with variant and wild-type GH diluted to a known standard range of concentrations (0.1-10 nM) in serum-free DMEM:F-12 (1:1) containing 2.5×10⁻⁷M dexamethasone for 6 hours to allow GHR dimerization, STAT5 activation and luciferase expression. After incubation, cells were lysed and the luciferase measured in a microplate luminometer (Applied Biosystems) using the Promega luciferase assay system. Luciferase expression thus provided a measure of the degree of GHR activation and hence the biological activity of the GH variant. Experiments were carried out in quadruplicate and repeated at least 3 times. Statistical analysis of luciferase assay data was carried out by analysis of variance (ANOVA) with subsequent comparisons using the Student-Newman-Keuls multiple comparison test

GH Secretion Studies in Mammalian Cells

The rat pituitary (GC) cell line was transfected with a pGEM-T plasmid containing a 3.2 kb fragment spanning the entire wild-type GH1 gene (under the control of promoter haplotype 1) and equivalent constructs for the missense variants under the control of their associated haplotypes. Cells were plated into 24-well plates (200,000 cells per well) and cultured overnight in DMEM containing 15% horse serum and 2.5% FCS (complete medium). Cells were co-transfected with 500 ng GH1 plasmid and β-galactosidase expression vector (pCH110; Amersham Pharmacia Biotech) using the lipid-based transfection reagent Tfx-20 (Promega). Transfection was carried out in 200 μl serum-free medium containing 1 μl Tfx-20/well for 1 hr, after which 0.5 ml complete medium was added to each well. Cells were cultured for 48 hrs, medium harvested and cells lysed for β-galactosidase assay to correct for differences in transfection efficiency. With the exception of Arg16Cys, GH in the medium was quantified for all variants using a human GH IRMA (Nichols Institute Diagnostics) that showed no cross-reactivity with rat GH. Owing to lack of cross-reactivity of the Arg16Cys variant in the GH IRMA, this variant was quantified using a human GH ELISA (DRG Diagnostics). The Arg16Cys variant fully cross-reacted in this assay, diluting out in parallel with the standard curve, whilst rat GH showed no cross-reactivity. Results for the Arg16Cys variant were compared to wild-type GH quantified using the ELISA kit in the same experiment. Experiments were performed and data analysed as described for the biological activity assay.

Functional Characterization of Missense Variants

Missense mutations in the mature protein were modelled by simple replacement of the appropriate amino acid residue in the X-ray crystallographic structure of human GH. The majority of missense mutations were found to be compatible with a model of structural deformation of the GH molecule (concomitantly impairing protein folding and hence reducing bioactivity), rather than with a model of a dysfunctional yet normally folded protein. However, three of the missense mutations (Arg16Cys, Lys41Arg, Thr175Ala) were located in regions of the GH molecule known to interact with the GHR (De Vos et al Science 255 306-12 (1992)). Indeed, two of the amino acids involved (Lys41 and Thr175) are among 8 key residues previously identified as being necessary for tight binding affinity between site 1 of GH and the GHR (Cunningham and Wells 234 554-63 (1993); Clackson and Wells Science 267 383-6 (1995); Wells Proc Natl Acad Sci USA 93 1-6 (1996)).

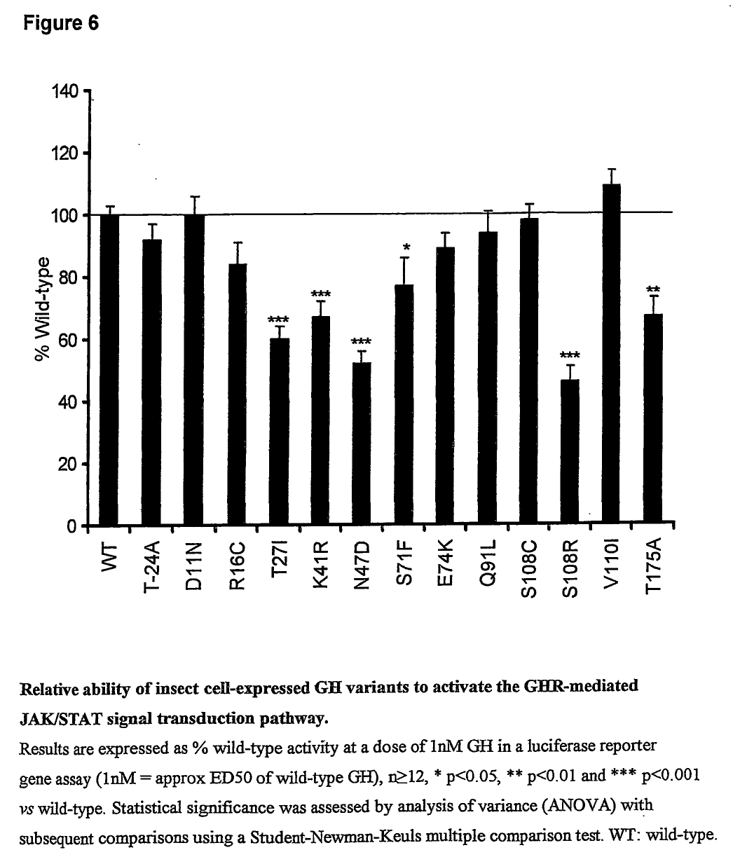

Thirteen of the GH missense variants were expressed in insect cells, the exception being Leu-11Pro which was not secreted into the culture medium. A luciferase reporter gene assay system was then used to assay their signal transduction activity. For GH to be biologically active, it must bind to two GHR molecules thereby triggering receptor dimerization. This then activates the intracellular tyrosine kinase JAK-2 which, in turn, activates the transcription factor STAT5 by phosphorylation. Phosphorylated STAT5 dimerizes, translocates to the nucleus and binds to STAT5-responsive promoters to switch on the expression of GH-responsive genes. The assay of GH biological activity used here requires all stages of this pathway to be functional. Six variants (Thr27Ile, Lys41Arg, Asn47Asp, Ser71Phe, Ser108Arg and Thr175Ala) were found to be associated with a significantly reduced ability to activate the JAK/STAT signal transduction pathway whereas the remaining seven (Thr-24Ala, Asp11Asn, Arg16Cys, Glu74Lys, Gln91Leu, Ser108Cys and Val110Ile) displayed normal or near normal functional activity (FIG. 6). In principle, the latter variants could either have exerted their deleterious effects on a signal transduction pathway other than JAK/STAT or their detrimental effects may not have been manifest in a static in vitro system. Alternatively, these variants could have compromised GH1 mRNA splicing, GH folding, secretion or stability, or may have exerted their adverse effects on the GH axis in other ways. Finally, they might quite simply have been rare neutral variants with no phenotypic effect.

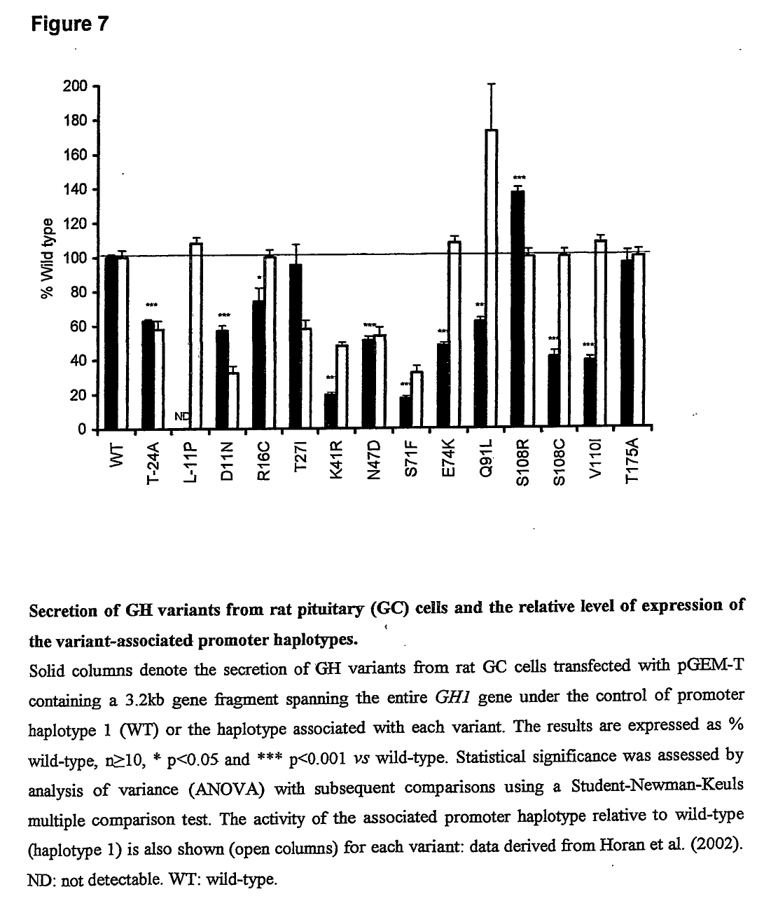

To further explore these possibilities, secretion studies of the GH missense variants were performed in rat pituitary GC cells. The wild-type GH1 gene, under the control of GH1 promoter haplotype 1, was transfected into GC cells and shown to be responsible for the secretion of human GH (as measured by IRMA using a human GH-specific antibody) at a concentration of 64 pM over a 48 hr period. Each GH variant was assayed under the control of its associated promoter haplotype with the GH secretion level measured being expressed as a percentage of wild-type (FIG. 7). Since reduced secretion with respect to wild-type may be either wholly or partially attributable to reduced expression resulting from the possession of a low expressing promoter haplotype rather than to the direct effect of the missense mutation, the empirically derived levels of expression for each associated proximal promoter haplotype were compared directly (FIG. 7). Although the amount of GH secreted by the Ala-24 variant was ˜63% that of the wild-type Thr-24, the associated promoter haplotype 21 exhibits only 58% of the promoter activity associated with the wild-type promoter haplotype 1. It may therefore be inferred that the Thr-24A mutation has little or no effect on GH secretion and that the reduced secretion manifested by the Ala-24-bearing allele is attributable solely to the presence in cis of a low expressing promoter haplotype. Although reduced promoter activity is probably also sufficient to account for the reduced secretion of the Asp11Asn and Asn47Asp variants, the low level of secretion of the functionally impaired Lys41Arg and Ser71Phe variants is probably not explicable solely in terms of the associated low expressing promoter haplotype. By contrast, a GH1 construct containing the Leu-1 Pro mutation secreted no measurable GH despite being associated with a normally expressing promoter (haplotype 2). Similarly, the reduced secretion manifested by variants Arg16Cys, Glu74Lys, Gln91Leu, Ser108Cys and Val100Ile could not be attributed to a low expressing promoter haplotype and is therefore likely to be a consequence of the introduced missense mutations. Together with the Leu-11Pro leader peptide mutation, these five variants therefore comprise a distinct group in that they compromise GH secretion rather than functional activity. Secretion of the Thr27Ile and Thr175Ala variants was comparable to the wild-type whilst that of the Ser108Arg was elevated.

A single example of a novel Val110Ile substitution was found among the individuals with short stature selected according to the aforementioned criteria. However, since four Ile110 alleles were also noted in the control group (corresponding to an allele frequency of 0.013), this variant may be regarded as a polymorphism in the general population. Molecular modelling suggested that this substitution might exert a deleterious effect on GH structure. Indeed, the evolutionarily conserved Val110 residue forms part of the hydrophobic core at the N-terminal end of helix 3, and its replacement by Ile with its longer side-chain would be expected to cause steric hindrance. Consistent with this prediction, a Val110Phe substitution has been reported as a cause of autosomal dominant type II IGHD (Binder et al J Clin Endocrin Metab 86 3877-81 (2001)). Since the Ile110 variant reported here exhibited significantly reduced secretion, it may be regarded as a functional polymorphism.

The adoption of the aforementioned criteria for clinical selection appears to have been instrumental in allowing us to detect novel GH1 gene lesions in the probands tested. Indeed, functionally significant mutations were found to occur significantly more frequently among selected (6/41) than among unselected individuals [7/154; odds ratio: 3.6, 95% confidence interval (CI): 1.0-12.9]. If the Val110→Ile functional polymorphism were excluded from this comparison, the odds ratio would be 7.0 (95% CI: 1.4-39.0). The prevalence of functionally significant GH1 gene lesions in the group was, however, significantly lower than in a group of IGHD patients without gross GH1 gene deletions (6/11; odds ratio: 25.2; 95% CI: 5.1-132.2). The successful use of the aforementioned criteria to enrich for novel GH1 gene mutations demonstrates that the identification of carriers of GH1 gene lesions may be achieved by reference to auxological parameters and bone age, irrespective of the results of GH secretion tests.

On the other hand, since probands found to possess a GH1 gene lesion did not differ significantly from non-carrier probands in terms of any measured laboratory or clinical phenotypic parameter, it is unlikely that many carriers could be readily identified among probands without recourse to the use of DNA sequencing as a screening technique.

Of the variants identified according to this Example, Leu-11Pro, Lys41Arg, Asn47Asp, Ser 71Phe, Ser108Arg, Thr175Ala, Glu74Lys, Gln91Leu, Ser108Cys and Val110Ile are described in our co-pending patent specification no. PCT/GB01/2126. The variants Thr 27Ile, which exhibits a reduced ability to activate the JAK/STAT pathway, and Arg16Cys, which reduces secretion in rat pituitary cells after allowance has been made for the level of expression attributable to the associated GH1 proximal promoter haplotype, and Ile179Met which exhibits a reduced ability to activate the MAP Kinase signal transduction pathway are described here for the first time, and, in the former two cases, referred to in FIGS. 6 and 7 as T27I and R16C, respectively.

### EXAMPLE 7

### Activation of MAP Kinase Pathway by Variation Ile179Met

Proteolytic Digestion of the GH Variant

Trypsin, chymotrypsin, or proteinase K (all Sigma, Poole, UK) were added to a final concentration of 0.1 μg/ml to 100 μl culture medium harvested from insect cells expressing either wild-type GH or the Ile179Met variant (60 nM) and then incubated at 37° C. for 1 hr. Previous dose-dependent studies on wild-type GH indicated that 0.1 μg/ml was the concentration at which degradation was initiated by all three enzymes. After the 1 hr treatment period, 10 μl trypsin-chymotrypsin inhibitor (500 μg/ml) was added to stop the trypsin and chymotrypsin digests and 1 μl PMSF (0.1M) was added to stop the proteinase K digest. Each reaction was then incubated for a further 15 mins at 37° C. The samples were analysed by SDS-PAGE on a 12% gel using a mini gel apparatus (Bio-Rad Laboratories). An equivalent amount of undigested wild-type GH and Ile179Met variant that had been incubated for 1 hr at 37° C. was also run on the gel. The gel was electroblotted onto PVDF membrane as previously described (Lewis et al Neuroendrocinology 2002. 14, 361-367), probed with a mouse monoclonal anti-human GH antibody (Lab Vision, Fremont, Calif., USA), diluted 1:500, detected using an anti-mouse IgG-HRP conjugate (1:5000, Amersham Biosciences) and visualised by enhanced chemiluminescence (ECL Plus, Amersham Biosciences). Films were analysed using the Alpha Imager 1200 digital imaging system (Alpa Innotech Corp, San Leandro, Calif., USA) and the results expressed as the amount of GH remaining following enzyme digestion as a percentage of undigested GH. The experiments were repeated 3 times and assessed statistically by a two-tailed t-test.

Activation of the MAP Kinase Pathway

The ability of the Ile179Met variant to activate the MAP kinase signal transduction pathway to the same degree as wild-type GH was investigated by stimulating 3T3-F442A preadipocytes with wild-type GH and the Ile179Met variant (20 nM for 15 mins). Cells were then lysed and analysed by SDS-PAGE on a 10% gel as described above. The gel was blotted onto PVDF membrane and probed using antibodies that detect the activated (phosphorylated) forms of p42/p44 MAP kinase (Cell Signaling Technology) and STAT 5 (Upstate Biotechnology). Blots were processed, visualised using ECL Plus (Amersham) and the images analysed as described above.

Functional Characterization of the Ile179Met Variant

The evolutionary conservation of the hydrophobic residue Ile179 was examined by ClustalW multiple sequence alignment of orthologous GH proteins from 19 vertebrates (Krawczak et al Gene 1999. 237,143-151). This residue is a hydrophobic valine in all vertebrates except turtle, indicating that the substitution by GH in the human lineage is conservative. Comparison with the paralogous genes of the human GH cluster revealed that the residue analogous to Ile179 is Met in CSH1, CSH2 and the CSH pseudogene (CSHP1). This is consistent with the conservative Ile179Met substitution having been templated by gene conversion.

The Ile79Met substitution was then modelled by replacement of the residue in the X-ray crystallographic structure of human GH. Ile179 lies in helix 4 where it is partially exposed, allowing hydrophobic interactions with the side-chain of the “hotspot” GHR residue Trp169. Further interactions with the GHR occur between the side-chain and backbone atoms of Ile179 and the backbone atoms of GHR residues Lys167 and Gly168. Replacement of the Ile179 side-chain with the side-chain of methionine indicates that these hydrophobic interactions may be conserved upon substitution.

The Ile179Met variant was expressed in insect cells and a luciferase reporter gene assay system (11, 12) used to assay its signal transduction activity. For GH to be biologically active, it must bind to two GHR molecules thereby triggering receptor dimerization. GHR dimerization activates the intracellular tyrosine kinase JAK2 which in turn activates the transcription factor STAT 5 by phosphorylation. Phosphorylated STAT 5 dimerizes, translocates to the nucleus and binds to STAT 5-responsive promoters thereby switching on the expression of GH-responsive genes. The assay of GH biological activity used here requires all stages of this pathway to be functional. The Ile179Met variant was found to display normal (99±4% wild-type) ability to activate the JAK/STAT signal transduction pathway.

However the above study designed to assess the ability of the Ile179Met variant to activate the MAP kinase pathway indicated a considerably reduced level of activation in response to the variant (5.7 times basal level of activation) as compared to wild-type (14.5 times basal level of activation). This contrasted with its ability to activate STAT 5 to the same level as wild-type GH [20.5 times for the wild-type (Ile179) versus 22.5 times for the Met179 variant]. The STAT 5 data confirmed the result from the STAT 5-responsive luciferase bioassay showing the same level of activity between wild-type GH and the Ile179Met variant.

To explore these possibilities further, the secretion of the Ile179Met variant was studied in rat pituitary GC cells. The wild-type GH1 gene, under the control of GH1 promoter haplotype 1, was transfected into GC cells and shown to be responsible for the secretion of human GH (as measured by RIA using a human GH-specific antibody) at a concentration of 64 pM over a 48 hr period. The Ile179Met variant (also under the control of GH1 promoter haplotype 1 with which it is associated in cis in patient B49) was then assayed, and the GH secretion level measured was expressed as a percentage of wild-type. Since secretion was found to be 97±4% of the wild-type value, it may be inferred that this mutation is likely to have little or no effect on GH secretion.

Finally, the Ile179Met variant was also challenged with trypsin, chymotrypsin and proteinase K to determine if it was more susceptible to proteolytic cleavage than wild-type GH. However, the 179Met variant proved similarly resistant to proteolytic cleavage as wild-type GH indicating that there were no significant differences in protein folding between the two forms of GH.

In this initial assessment we examined the ability of the Ile179Met variant to activate the JAK/STAT signal transduction pathway and found it to be indistinguishable from wild-type. Secretion and stability of this variant also appeared to be normal. We then examined the ability of the variant to activate the MAP kinase signal transduction pathway and found it to be significantly decreased. We believe therefore that this variant is dysfunctional in that it manifests reduced ability to activate the MAP kinase signal transduction pathway. Accordingly, it represents another important variant of the Growth Hormone gene that is likely to exhibit normal immunological reactivity but no biological activity.

1. An isolated or recombinant polynucleotide comprising a variant of the human growth hormone nucleic acid sequence, GH1, which variant comprises a variation selected from the group consisting of:
(a)
(i) +480 C→T;
(ii) +446 C→T;
(iii) +1491 C→G;
(iv) −60 G→A;
(v) −40 to −39 GG→CT;
(vi) −360 A→G; and
(vii) +748 A→G
(where figures relate to reference wild-type human GH1 nucleotide position number, counting from TSS); and
(b) an oligonucleotide specific for the sequences (a) and comprising a variation selected from (i) to (vii).

2. A nucleic acid sequence according to claim 1, wherein the sequence (a) is selected from:
(a)
(i) +480C→T; and
(ii) +446C→T.

3. A nucleic acid sequence according to claim 1, which is a cDNA sequence.

4. An amino acid sequence encoded by a variant of GH1, wherein the variant of GH1 is one according to claim 1.

5. A human GH variant, defined with reference to hGH (FIG. 5, SEQ ID NO:), selected from:
(i) Thr27Ile;
(ii) Arg16Cys;
(iii) Ile179Met;
(iv) Thr27Ile; and
(v) Asn47Asp.

6. A variant according to claim 4, which comprises Thr27Ile.

7. A variant according to claim 4, which comprises Arg16Cys.

8. A variant according to claim 4, which comprises Ile179Met.

9. An in vitro screening method for screening a patient suspected of having dysfunctional GH, which screening method comprises the steps of:
(a) obtaining a test sample comprising a nucleotide sequence of the human GH1 gene or a polypeptide sequence encoded thereby from the patient; and
(b) comparing a region of the sequence obtained from the test sample with the corresponding region of a predetermined sequence characterized in that the predetermined sequence is selected from a variant of GH1 according to claim 1, or an amino acid sequence encoded by the variants of claim 1.

10. A screening method according to claim 9, wherein the predetermined sequence is an oligonucleotide.

11. A screening method according to claim 9, wherein the test sample comprises genomic DNA.

12. A screening method according to claim 9, wherein the comparison step includes the step of sequencing the appropriate region of the GH1 gene and/or employs DNA chip technology wherein the chip is a miniature parallel analytical device that is used to screen simultaneously either for multiple known mutations or for all possible mutations, by hybridisation of labelled sample DNA.

13. A screening method according to claim 9, wherein the comparison step comprises identification of the polypeptide by protein sequencing methods, including mass spectroscopy, micro-array analysis and pyrosequencing and/or antibody-based methods of detection, including ELISA.

14. A kit suitable for use in carrying out a screening method according to claim 9, which kit comprises:
(a) an oligonucleotide having a nucleic acid sequence corresponding to a region of a variant GH1 gene, which region incorporates at least one variation selected from the group consisting of:
(i) +480 C→T;
(ii) +446 C→T
(iii) +1491 C→G;
(iv) −60 G→A;
(v) −40 to −39 GG→CT;
(vi) −360 A→G;
(vii) +748 A→G
(where figures relate to reference wild-type human GH1 nucleotide position number, counting from TSS); and an oligonucleotide specific for the sequences (i) to (vii) and comprising a variation selected from (i) to (vii).
(b) an oligonucleotide having a nucleic acid sequence corresponding to the wild-type sequence in the region specified in (a); and, optionally,
(c) one or more reagents suitable for carrying out PCR for amplifying desired regions of the patient's DNA.

15. A kit according to claim 14, wherein the reagent(s) comprise one or more of: PCR primers corresponding to an exon of the GH1 gene, and/or primers defined hereinabove; and/or other reagents for use in PCR, including Taq DNA polymerase.

16. A screening method according to claim 9 employing one or more ‘surrogate marker(s)’ that are indicative of or correlated to the presence of a variant of GH1 comprising a variation selected from the group consisting of:
(i) +480 C→T;
(ii) +446 C→T;
(iii) +1491 C→G;
(iv) −60 G→A;
(v) −40 to −39 GG→CT;
(vi) −360 A→G;
(vii) +748 A→G
(where figures relate to reference wild-type human GH1 nucleotide position number, counting from TSS); and an oligonucleotide specific for the sequences (i) to (vii) and comprising a variation selected from (i) to (vii).

17. A screening method or kit according to claim 16, wherein the ‘surrogate marker’ is or includes:
(a) any biomolecule (including, but not limited to, nucleotides, proteins, including antibodies specific for the GH variant or the variant of GH1, sugars and lipids);
(b) a chemical compound (including, but not limited to, drugs and metabolites thereof); and/or
(c) a physical characteristic,
whose absence, presence, or quantity in an individual is measurable and correlated with the presence of the GH variant or the variant of GH1.

18. The use of a variant of GH1 comprising a variation selected from the group consisting of:
(i) +480 C→T;
(ii) +446 C→T;
(iii) +1491 C→G;
(iv) −60 G→A;
(v) −40 to −39 GG→CT:
(vi) −360 A→G;
(vii) +748 A→G
(where figures relate to reference wild-type human GH1 nucleotide position number, counting from TSS); and an oligonucleotide specific for the sequences (i) to (vii) and comprising a variation selected from (i) to (vii) in a therapeutic, diagnostic or detection method.

19. The use according to claim 18 for the determination of susceptibility of an individual to a disease selected from diabetes, obesity, infection, cancers or cardiac conditions.

20. The use according to claim 18 for the determination of GH binding defects and/or pituitary storage defects in an individual.

21. The use of a variant of GH1 according to claim 1 in gene therapy.

22. The use of a variant of GH1 comprising a variation selected from the group consisting of:
(i) +480 C→T;
(ii) +446 C→T;
(iii) +1491 C→G;
(iv) −60 G→A;
(v) −40 to −39 GG→CT;
(vi) −360 A→G;
(vii) +748 A→G
(where figures relate to reference wild-type human GH1 nucleotide position number, counting from TSS); and an oligonucleotide specific for the sequences (i) to (vii) and comprising a variation selected from (i) to (vii) the preparation of a therapeutic composition, diagnostics composition or kit, or detection kit for preventing, treating, diagnosing or detecting conditions associated with or caused by GH dysfunction in an individual.

23. An antibody specific for a variant according to claim 4, which antibody is capable of distinguishing between the variant and corresponding wild type amino acids.

24. A composition comprising a GH variant according to claim 4 in association with a pharmaceutically acceptable carrier therefor.

25. A vector comprising a nucleic acid sequence according to claim 1.

26. A host cell comprising a vector according to claim 25, including a bacterial host cell.

27. A process for preparing a GH variant, which process comprises:
(i) culturing a host cell according to claim 26; and
(ii) recovering from the culture medium the GH variant thereby produced.

28. A protein or amino acid sequence encoded or expressed by a sequence, vector, or cell according to the variants described in claim 1 which protein or amino acid sequence is in culture medium.
